(19) United States
(12) Patent Application Publication
(10) Pub. No.: US 2007/0254002 A1
(43) Pub. Date: Nov. 1, 2007

(54) Biocompatible devices coated with activated protein C

(76) Inventor: Sheng-Qian Wu, Brighton, MA (US)

Correspondence Address: CLARK & ELBING LLP, 101 FEDERAL STREET, BOSTON, MA, 02110, US

(21) Appl. No.: 11/411,614

(22) Filed: Apr. 26, 2006

(51) Int. Cl. A61F 2/02; C08G 63/48

(52) U.S. Cl. 424/423; 424/445; 525/54.1

(57) ABSTRACT

The invention features a composition, such as a medical device, that is coated with activated protein C (APC).

### FIELD OF THE INVENTION

The invention relates to modification of medical devices to prevent thrombosis.

### BACKGROUND OF THE INVENTION

Polyester (DACRON® or polyethylene terephthalate) fibers were first characterized in 1941 and have become the most widely produced synthetic fiber in the world. They are most familiarly known by the DuPont commercial name DACRON®. The polymer is synthesized by a condensation reaction of derivatives of ethylene glycol and terephthalic acid, resulting in molecules that contain 80 to 100 repeat units. These molecules are then extruded through a plurality of holes (a spinneret) to produce multi-filament fibrous filaments. Such DACRON® fibers are further processed into various structures such as warp-knit, weft-knit, and woven fabrics that have excellent resiliency as well as resistance to a wide range of chemical and biological challenges.

DACRON® is utilized in items ranging from clothing to medical implants. DACRON® yarn was first sewn into a tubular form and utilized as a large-diameter vascular graft in the mid-1950s. Since that point, DACRON® has been incorporated into both large and medium bore vascular grafts in knitted and woven form. These grafts have shown excellent long-term biodurability, handling characteristics and capsular tissue incorporation.

Polyester is known as a relatively inert fiber. It is hydrophobic, both in bulk and in its surface properties. At normal temperatures it has low uptake of moisture, dyes and other chemicals. In normal textile use, it tends to suffer associated disadvantages: it generates static electricity, it does not readily shed oily soils, and it does not wet enough to encourage the wicking of water. For applications where repellency is required, however, it is insufficiently hydrophobic, and repellent finishes are applied. Like any fiber, softness is advantageous, and chemical softeners are applied.

Modifications to overcome these deficiencies typically rely on the surface deposition of polymeric textile finishes. These include silicones, vinyl and acrylic polymers, and fluorochemicals. Other finishes are based on an ester interchange reaction that fixes a hydrophilic moiety (typically a short chain polyethylene oxide). Many of these finishes suffer a lack of durability to laundering and dry cleaning, since (other than those bonded via ester interchange) they are not covalently bonded to the polyester surface.

Polyester is employed for various medical devices such as prosthetic vascular grafts, prosthetic heart valve sewing cuffs, left ventricular assist devices, artificial organs, wound patches and wound dressings. Polyester is a biodurable material due to the relatively inert properties of the polymer and can persist for greater than 10 years when implanted without significant deleterious effects to the specific device. However, this material, similar to other biomaterials, is prone to 3 major complications when implanted: 1) thrombosis (clot formation), 2) infection and 3) lack of cell appropriate healing. These adverse properties occur as a result of the bulk properties of the polymer. Additionally, the rigidity of the polymer limits surface modification in order to incorporate various moieties such as anti-thrombolytic agents (e.g., anti-thrombin), thrombolytic agents, growth-promoting factors, growth-inhibiting factors, and antimicrobial/antifungal agents.

A complication of all implantable biomaterials is incompatibility between blood and the biomaterial surface. The initial interaction of blood and the foreign surface results in an array of activation or biologic responses: platelet activation and adhesion, activation of the intrinsic pathway of the coagulation cascade resulting in formation of active thrombin, leukocyte activation and the release of complement and kallikrein. If unregulated, these responses lead to surface thrombus formation with subsequent failure of the implanted biomaterial.

Numerous attempts have been made to create a more biocompatible surface by establishing a new biologic lining on the luminal surface that would “passivate” this acute initial reaction. These efforts have ranged from non-specifically binding albumin to the surface followed by heat denaturation to non-specifically crosslinking albumin, gelatin and collagen. Covalent or ionic binding of the anticoagulant heparin alone, in conjunction with other biologic compounds, or with spacer moieties as well as covalent linkage of thrombomodulin have also been performed. Other studies have focused on modifying the composition of the biomaterial by either increasing hydrophilicity via incorporation of polyethylene oxide groups or by creating an ionically charged surface.

Each of these methodologies has had limited success in creating a durable, biologically-active surface. There are several limitations associated with these surface modifications: 1) thrombin is not directly inhibited therefore fibrinogen amounts remain constant on the material surface permitting platelet adhesion, 2) heparin-coated biomaterials may be subject to heparitinases limiting long-term use of these materials, 3) non-specifically bound compounds are desorbed from the surface which is under shear stresses thereby re-exposing the thrombogenic biomaterial surface, 4) rapid release of non-specifically bound compounds may create an undesired systemic effect and 5) charge-based polymers may be covered by other blood proteins such that anticoagulant effects are masked.

Endothelial cells play a pivotal role in mediating blood interaction with the arterial wall. These cells maintain hemostasis and also synthesize growth mediators that block abnormal smooth muscle cell proliferation. Ideally, prosthetic grafts should promote endothelial cell adherence and growth on the luminal surface while permitting direct host tissue incorporation at the capsular surface. This type of cellular incorporation does not occur in actuality, thereby predisposing these grafts to infection, thrombosis, perigraft seromas and delayed graft failure. Thus, failure of appropriate cell type growth and development to these biomaterials significantly limits their expanded use.

To avert these complications and mimic the non-thrombogenic in vivo endothelial cell blood vessel lining, cell adhesion to prosthetic grafts using endothelial cell seeding techniques have been extensively employed. Adhesive proteins such as fibronectin, fibrinogen, vitronectin and collagen have served well in graft seeding protocols. The cell attachment properties of these matrices can also be duplicated by short peptide sequences such as RGD (Arg-Gly-Asp). These adhesive proteins, however, have several drawbacks: 1) bacterial pathogens recognize and bind to these sequences, 2) non-endothelial cell lines also bind to these sequences, 3) patients requiring a seeded vascular graft have few donor endothelial cells, therefore cells must be grown in culture and 4) endothelial cell loss to shear forces remains a significant obstacle.

Modification of the surface has also been employed to modify host response to the foreign body, serving as an approach for improving endothelial cell adherence to DACRON®. Endothelial cells after seeding have been shown to attach and grow on a variety of protein substrates coated onto vascular graft materials. Bioactive oligopeptides and cell growth factors have been immobilized onto various polymers and demonstrated to affect cell adherence and growth. Additional studies have described the incorporation of growth factors into a degradable protein mesh, resulting in the formation of capillaries into the graft wall. Utilizing these techniques to incorporate growth factors, however, does have major limitations: 1) growth factor is rapidly released from the matrix, 2) matrix degradation re-exposes the thrombogenic surface, thus endothelialization is not uniform and 3) release of non-endothelial specific growth factor is not confined to the biomaterial matrix, thereby exposing the “normal” distal artery to the growth factor.

There have been several studies assessing the effects of amine interaction with polyester. Zahn et al. (Polymer 3:429, 1962), as well as Farrow et al. (Polymer 3:17, 1962) assessed the lysis of polyester in an attempt to breakdown excess material in the textile industry into smaller components, without regard to maintaining the integrity of the polymer structure. In 1982, Ellison et al. examined the effects of a monofunctional amine versus alkali hydrolysis on polyester. These studies, which again were performed under harsh conditions, demonstrated that alkaline hydrolysis has a more substantial effect on fiber weight without extensive strength loss. In contrast, aminolysis had less effect on fiber weight but a greater effect on fiber strength, indicative of a permanent reaction within the polymer structure. In 1968, Avny and Rebenfeld demonstrated that multi-functional amine compounds could be reacted within the polymer structure (three or more amine groups) while presenting minimal loss in strength (Applied Polymer Science 32:4009, 1986).

Activated protein C (APC) is a natural potent anticoagulant. The vitamin K-dependent glycoprotein-protein C is activated on the endothelial cell surface by the thrombin/thrombomodulin (TM) complex in a process that is stimulated approximately 20-fold by the endothelial protein C receptor (EPCR). Once activated, APC cleaves and inactivates factors Va and VIIIa in a reaction that is accelerated by the cofactor, protein S. The importance of protein C/protein S and TM in mediating blood fluidity is evidenced by the observation that congenital absence or deficiency of these proteins in animal models and humans results in increased risk for thrombosis. In addition to inactivating factors Va and VIIIa, APC has been shown to attenuate fibrin deposition, accelerate fibrinolysis, reduce neutrophil chemotaxis and graft neutrophil activation.

There is increasing evidence that APC signals in cells through a poorly understood mechanism that involves EPCR and the thrombin receptor-PAR-1. APC has been shown to activate the thrombin receptor, PAR-1, through an EPCR-dependent mechanism in endothelial cells, resulting in a significantly weaker signal compared to that of thrombin. APC has also been shown to have anti-platelet activation, anti-inflammatory, antiapoptotic, and anticoagulant activity at the cellular level; in vitro studies have shown that APC induces endothelial cell proliferation and migration. APC is a natural ligand for EPCR, which is expressed primarily on endothelial cells.

There remains a need for suitable medical device materials and coatings that provide functional moieties for attachment of commercial finishes or biologically-active agents, while retaining material strength.

### SUMMARY OF THE INVENTION

A first aspect of the invention features a biocompatible medical device that includes, immobilized to the exterior of the device, activated protein C (APC). In a preferred embodiment, the device includes a polyethylene terephthalate polymer that includes a carboxylic acid group derived from a terephthalate moiety of the polymer and an amine group derived from reaction of the polymer with a diamine. According to this embodiment, the APC is immobilized to the polyethylene terephthalate polymer by a covalent bond via the carboxylic acid group or the amine group. In other embodiments, the carboxylic acid group results from hydrolysis of the terephthalate moiety, the diamine is selected from ethylene diamine (EDA), 2-methylpentamethylene diamine, 1,2-diaminocyclohexane, and 1,6-hexanediamine, and the polyethylene terephthalate polymer is treated by alkaline hydrolysis prior to reaction with the diamine.

In other embodiments, the device can be selected from a stent, a catheter, a vascular graft, an artificial heart, a blood filter, a pacemaker lead, a heart valve, and a prosthetic graft. In yet other embodiment, the coating of the device additionally includes a chemical compound (e.g., a commercial finish selected from the group consisting of flame retardants, repellents, antistatic agents, and dyes) or a biologically-active agent (e.g., an antimicrobial agent, an antifungal agent, an anti-thrombolytic agent, a thrombolytic agent, an antiviral agent, an antiseptic agent, a growth-promoting agent, or a growth-inhibiting agent). In another preferred embodiment, the biologically-active agent is a peptide, a polypeptide (e.g., anti-thrombin, fibronectin, fibrinogen, vitronectin, collagen, streptokinase, urokinase, tissue plasminogen activator (tPA), vascular endothelial growth factor (VEGF), or gamma-interferon), a nucleic acid molecule, an antibody, or a small molecule.

A second aspect of the invention features a composition that includes a biocompatible material with APC coated to the material. In an embodiment, the APC is coated on an external surface of the composition. In other preferred embodiments, the biocompatible material includes a bifunctionalized polyethylene terephthalate polymer having a carboxylic acid group derived from a terephthalate moiety of the polymer and an amine group derived from reaction of the polymer with a diamine. According to this embodiment, the APC is immobilized to the polyethylene terephthalate polymer by a covalent bond via the carboxylic acid group or the amine group. In other embodiments, the carboxylic acid group results from hydrolysis of the terephthalate moiety, the diamine is selected from ethylene diamine (EDA), 2-methylpentamethylene diamine, 1,2-diaminocyclohexane, and 1,6-hexanediamine, and the polyethylene terephthalate polymer is treated by alkaline hydrolysis prior to reaction with the diamine.

In other embodiments of the second aspect, the composition can be selected from a stent, a catheter, a vascular graft, an artificial heart, a blood filter, a pacemaker lead, a heart valve, a prosthetic graft, and a wound dressing. In yet other embodiment, the composition coating additionally includes a chemical compound (e.g., a commercial finish selected from the group consisting of flame retardants, repellents, antistatic agents, and dyes) or a biologically-active agent (e.g., an antimicrobial agent, an antifungal agent, an anti-thrombolytic agent, a thrombolytic agent, an antiviral agent, an antiseptic agent, a growth-promoting agent, or a growth-inhibiting agent). In another preferred embodiment, the biologically-active agent is a peptide, a polypeptide (e.g., anti-thrombin, fibronectin, fibrinogen, vitronectin, collagen, streptokinase, urokinase, tissue plasminogen activator (tPA), vascular endothelial growth factor (VEGF), or gamma-interferon), a nucleic acid molecule, an antibody, or a small molecule.

A third aspect of the invention features a method of generating a functionalized polyester material. This method includes incubating the polyester material with ethylene diamine in solution (aqueous or organic) under conditions that result in functionalization of the polyester material. The functionalization of the material consists of creation of carboxylic acid and amine groups within the polyester backbone. The carboxylic acid and amine groups can then be used as sites to attach other chemical compounds and biologically-active agents to the polyester backbone via an ionic or covalent bond. In a preferred embodiment, the biologically-active agent is activated protein C.

In a desired embodiment, the chemical compound consists of a commercial finish selected from the group consisting of flame retardants, repellents, antistatic agents, and dyes.

In several desired embodiments, the biologically-active agent applied to the bifunctionalized polyester polymer is desirably a small molecule (e.g., an organic compound with a mw<1000), but can also include, for example, a peptide, a polypeptide, a protein, a nucleic acid molecule, or an antibody. The biologically-active agent can act as an antimicrobial agent, an antifungal agent, an anti-thrombolytic agent (e.g., anti-thrombin and activated protein C), a thrombolytic agent, an antiviral agent, an antiseptic agent, an antibiotic, a growth-inhibiting agent, a growth-promoting agent, or a combination thereof. The antibiotic used in the method can include quinolone. Inorganic therapeutically-active compounds such as silver, silver salts, gold, or gold salts may also be bonded to the polymers of the present invention. This bonding may involve a covalent or an ionic interaction between the compound and the carboxylic acid group or amine group of the bifunctionalized polyester polymer.

The bifunctionalized polyester polymer which has bound an effective amount of the therapeutic compound or biologic agent can be used in any medical application in which biocompatible polymers are used (e.g., a biocompatible device), and in which infection or other complications are to be avoided. Examples include, but are not limited to, use as a wound dressing or implantable device. Desired devices are catheters, vascular grafts, artificial hearts, other artificial organs and tissues, blood filters, pacemaker leads, heart valves, and prosthetic grafts. The bifunctional polyester material, when used in vascular grafts, should not activate coagulation or inhibit cellular healing, is desirably biodurable, non-thrombogenic, chemically durable, resistant to infection or formation of microbial plaques, easy to implant, and possesses appropriate elastic properties. The bifunctional polyester material should also be sufficiently malleable so that it can form the appropriate geometry, but also have sufficient tensile strength to endure rigorous circulation throughout the vascular tree. The surface properties of the graft can be modified with biologically-active proteins in order to emulate certain natural properties of native vessels, thereby improving graft patency and healing. For instance, anti-thrombin (recombinant hirudin) or other anti-clotting agents, thrombolytic agents (e.g. streptokinase, urokinase, tissue plasminogen activator (tPA), pro-urokinase, etc.), and mitogenic agents (e.g. vascular endothelial growth factor) or other growth promoting substances, or inhibitors (e.g. γ-interferon) can be linked to the surface of the graft.

The biocompatible material should be able to be sterilized, for example, by gamma radiation.

In various embodiments, the bifunctionalized polyester polymer is non-toxic, does not contain an exogenous binder agent, and/or does not induce clot formation. The bifunctionalized polyester polymer can also be used in commercial products that are desirably antibacterial, antiviral, or antifungal (e.g., shower curtains, clothing, and foam cushions).

### BRIEF DESCRIPTION OF THE DRAWINGS

FIG. 1 is a schematic showing the hydrolysis and aminolysis of polyester.

FIGS. 2A-2G are a series of photographs of scoured ethylene diamine-treated polyester segments in which the uptake of methylene blue indicates the presence of carboxylic acid groups. Polyester segments are treated with 100% EDA for 80 minutes, rinsed in water for 5 min. (FIG. 2A), 10 min. (FIG. 2B), 20 min. (FIG. 2C), 40 min. (FIG. 2D), 120 min. (FIG. 2E), 240 min. (FIG. 2F), or overnight (16.25 hours; FIG. 2G), and exposed to methylene blue at pH 8.

FIGS. 3A-3G are a series of photographs of scoured ethylene diamine-treated polyester segments in which the uptake of acid red indicates the presence of amine groups. Polyester segments are treated with 100% EDA for 80 minutes, rinsed in water for 5 min. (FIG. 3A), 10 min. (FIG. 3B), 20 min. (FIG. 3C), 40 min. (FIG. 3D), 120 min. (FIG. 3E), 240 min. (FIG. 3F), or overnight (16.25 hours; FIG. 3G), and exposed to acid red at pH 4.

FIGS. 4A-4E are a series of photographs of scoured ethylene diamine-treated polyester segments in which the uptake of methylene blue indicates the presence of carboxylic acid groups. Polyester segments are treated with 100% EDA (FIG. 4A), 90% EDA in water (FIG. 4B), 80% EDA in water (FIG. 4C), 70% EDA in water (FIG. 4D), or 50% EDA in water (FIG. 4E) for 80 minutes, rinsed in water for 10 min., and exposed to methylene blue at pH 8.

FIGS. 5A-5E are a series of photographs of scoured ethylene diamine-treated polyester segments in which the uptake of acid red indicates the presence of amine groups. Polyester segments are treated with 100% EDA (FIG. 5A), 90% EDA in water (FIG. 5B), 80% EDA in water (FIG. 5C), 70% EDA in water (FIG. 5D), or 50% EDA in water (FIG. 5E) for 80 minutes, rinsed in water for 10 min., and exposed to acid red at pH 4.

FIGS. 6A-6F are a series of photographs of scoured ethylene diamine-treated polyester segments in which the uptake of methylene blue indicates the presence of carboxylic acid groups. Polyester segments are treated with 100% EDA (FIG. 6A), 90% EDA in toluene (FIG. 6B), 80% EDA in toluene (FIG. 6C), 70% EDA in toluene (FIG. 6D), 50% EDA in toluene (FIG. 6E), or 25% EDA in toluene (FIG. 6F), for 80 minutes, rinsed in water for 10 min., and exposed to methylene blue at pH 8.

FIGS. 7A-7F are a series of photographs of scoured ethylene diamine-treated polyester segments in which the uptake of acid red indicates the presence of amine groups. Polyester segments are treated with 100% EDA (FIG. 7A), 90% EDA in toluene (FIG. 7B), 80% EDA in toluene (FIG. 7C), 70% EDA in toluene (FIG. 7D), 50% EDA in toluene (FIG. 7E), or 25% EDA in toluene (FIG. 7F), for 80 minutes, rinsed in water for 10 min., and exposed to acid red at pH 4.

FIG. 8A-8F are a series of photographs of hydrolysed ethylene diamine-treated polyester segments in which the uptake of methylene blue indicates the presence of carboxylic acid groups. Polyester segments are treated with 100% EDA (FIG. 8A), 90% EDA in toluene (FIG. 8B), 80% EDA in toluene (FIG. 8C), 70% EDA in toluene (FIG. 8D), 50% EDA in toluene (FIG. 8E), or 25% EDA in toluene (FIG. 8F), for 80 minutes, rinsed in water for 10 min., and exposed to methylene blue at pH 8.

FIGS. 9A-9F are a series of photographs of hydrolysed ethylene diamine-treated polyester segments in which the uptake of acid red indicates the presence of amine groups. Polyester segments are treated with 100% EDA (FIG. 9A), 90% EDA in toluene (FIG. 9B), 80% EDA in toluene (FIG. 9C), 70% EDA in toluene (FIG. 9D), 50% EDA in toluene (FIG. 9E), or 25% EDA in toluene (FIG. 9F), for 80 minutes, rinsed in water for 10 min., and exposed to acid red at pH 4.

FIGS. 10A-10G are a series of photographs of ethylene diamine-treated polyester segments in which the uptake of methylene blue indicates the presence of carboxylic acid groups. Polyester segments are treated with 100% EDA for 80 minutes, rinsed in toluene for 5 min. (FIG. 10A), 10 min. (FIG. 10B), 20 min. (FIG. 10C), 40 min. (FIG. 10D), 120 min. (FIG. 10E), 240 min. (FIG. 10F), or overnight (16.25 hours; FIG. 10G), and exposed to methylene blue at pH 8.

FIGS. 11A-11G are a series of photographs of ethylene diamine-treated polyester segments in which the uptake of acid red indicates the presence of amine groups. Polyester segments are treated with 100% EDA for 80 minutes, rinsed in toluene for 5 min. (FIG. 11A), 10 min. (FIG. 11B), 20 min. (FIG. 11C), 40 min. (FIG. 11D), 120 min. (FIG. 11E), 240 min. (FIG. 11F), or overnight (16.25 hours; FIG. 11G), and exposed to acid red at pH 4.

FIG. 12 is a photograph showing the uptake of CI Acid Red 1 or Methylene Blue dye after treatment of polyester fabric (hydrolyzed or unhydrolyzed) at 85° C. with 2-methylpentamethylene diamine (2 MPD) for 10 minutes, tetraethylenepentamine (TEP) for 20 minutes, 1,2-diaminocyclohexane (12 DC) for 2 hours, and 1,6-hexanediamine (16 HD) for 24 hours.

FIG. 13 is a photograph showing the uptake of CI Acid Red 1 dye after treatment of polyester fabric with varying concentrations of EDA in toluene for 20 hours at 50° C.

FIG. 14 is a photograph showing the uptake of Acid Red 1 or Methylene Blue dye into hydrolyzed or unhydrolyzed polyester fabric as a consequence of immersion time in EDA.

FIG. 15A and 15B are graphs showing the concentration of amino (FIG. 15A) and carboxylic acid (FIG. 15B) groups after EDA treatment.

FIGS. 16A-16D are photographs at either 500×, 850×, 1500× or 2000× magnification, respectively, showing the cracking of polyester fibers.

FIG. 17 is a graph showing the loss of tensile strength of EDA-treated polyester.

FIGS. 18A and 18B are graphs showing the wicking performance of EDA-treated polyester by weight (FIG. 18A) and by height (FIG. 18B).

FIG. 19 is a drawing of C.I. Acid Yellow 4 dye.

FIG. 20 is a graph showing the binding of ¹²⁵I-BSA to EDA-modified DACRON® using amine groups.

FIG. 21 is a graph showing the binding of ¹²⁵I-BSA to EDA-modified DACRON® using carboxylic acid groups.

FIG. 22 is a graph showing the binding of ¹²⁵I-BSA and of ¹³¹I-BSA to EDA-modified DACRON® using both amine and carboxylic acid groups.

FIG. 23 is a schematic showing that the reaction of Traut's reagent with a primary amine-containing molecule results in a modification in the amine-containing molecule that produces a terminal sulfhydryl group.

FIG. 24 is a schematic showing that the reaction of APC with Sulfo-SMCC produces a maleimide-activated APC, which can be further reacted with Dacron to produce Dacron containing surface-bound APC.

FIG. 25 is a photograph showing that the exposure of woven polyester to EDA resulted in the formation of both amine groups and carboxylic acid groups as indicated by AR-1 and MB staining, respectively.

FIG. 26A is a graph showing the effect on APC enzymatic activity following reaction of APC and sulpho-SMCC at different molar ratio. The in solution enzymatic activity of APC remained unchanged unless the molar ratio was greater than 1:10.

FIG. 26B is a graph showing the effect on APC clotting activity following reaction of APC and sulpho-SMCC. Ten ng of modified APC (at the indicated molar ratio) was used for the clotting assay. The anticoagulant activity remained unchanged at a molar ratio 1:2 compared to native APC (ratio 1:0) and decreased when the molar ratio was greater than 1:10.

FIG. 26C is a graph showing the effect on clotting time of Sulfo-SMCC modified APC. Sulfo-SMCC modified APC is capable of inhibiting blood clotting as indicated by the prolonged clotting time using both human and canine plasma.

FIG. 27A is a graph showing that Traut's activated EDA-Dacron (TED) significantly increased APC-SMCC binding to surface compared to plain Dacron (D).

FIG. 27B is a graph showing that, when surface-bound, APC activity increased with increasing APC:SMCC ratio.

FIG. 28A is a schematic representation of a simulated arterial flow system used to evaluate the biostability of surface bound APC.

FIG. 28B is a graph showing that under simulated arterial flow conditions, no significant loss of surface-bound APC was seen up to 17 days.

FIG. 29 is a graph showing that immobilization of APC renders surface anticoagulant activity as indicated by the prolonged clotting time in TED compared with control Dacron.

### DETAILED DESCRIPTION

The present invention features compositions (e.g., medical devices) coated with activated protein C (APC). Immobilization of APC on the surface of, e.g., medical devices, such as grafts and stents, prevents thrombogenic activity and reduces the risk of thrombosis upon implantation of the medical device. APC also promotes endothelial cell growth and protects endothelial cells from apoptosis. Thus, compositions, such as medical devices, that are coated with APC maintain both anti-thrombogenic and pro-endothelial cell growth activity. Compositions coated with APC demonstrate improved compatibility and performance when implanted in, e.g., the vascular system of a patient.

The present invention also features the use of a bifunctional amine compound, ethylene diamine (EDA), under select conditions (i.e., solution type, concentration, surface treatment) to establish both amine functional groups and carboxylic acid groups within the polyester backbone. The groups can then be reacted with various crosslinking agents, binders, catalysts or via direct ionic interaction with other moieties such as commercial finishes (i.e., flame retardants, repellents, anti-static agents, dyes) or biologically-active agents (i.e. anti-thrombolytic agents (e.g., anti-thrombin and APC), thrombolytic agents, growth-promoting/inhibiting agents, antimicrobial or antifungal agents). The bifunctionalized polyester fiber can be provided for use in medical and textile applications.

Binding to Functional Groups

The treatment of polyester using this method establishes a bifunctional surface onto which various agents, such as growth factors, anti-thrombolytic agents (e.g., anti-thrombin and activated protein C), thrombolytic agents, or antibiotics, can be bound by ionic or covalent interactions, either alone or in combination. Several methods can be employed for application of various finishes and biologic moieties to the surface. For finishes, functional groups capable of reacting with amine moieties include epoxy, isocyanate, methylol, fluoro- and chlorotriazine, vinyl sulphone and the like. These reactive groups can be attached to substances capable of increasing or decreasing the hydrophobic nature of the polyester surface, and thus modifying the static, wicking, softness and repellency properties of the fiber. Because these substances are thereby covalently bonded to the polyester, their durability is substantially increased. For biologic moieties such as anti-thrombolytic agents (e.g., anti-thrombin), thrombolytic agents, and growth-promoting/inhibiting agents, various crosslinking techniques employing homo- and heterobifunctional crosslinkers can be utilized. Additionally, various inorganic and organic catalysts, reactive agents (i.e. glutaraldehyde) or binder agents can be used. Lastly, the ionic or hydrophilic properties of the material could be exploited to incorporate various antimicrobial/antifungal agents or biologic agents to the surface.

The presence of reactable amine groups on the polyester surface permits attachment thereto of a range of materials. Lewin et. al. outlined some of the possible reactions and functionalities: functional groups capable of reacting with amine moieties include epoxy, isocyanate, methylol, fluoro- and chlorotriazine, vinyl sulphone and the like (Lewin et al., Handbook of fiber science and technology, Vol. 2 Part B, New York, Marcel Dekker Inc. 1984). These reactive groups can be attached to substances capable of increasing or decreasing the hydrophobic nature of the polyester surface, and thus modifying the static, wicking, softness and repellency properties of the fiber. Because these substances are thereby covalently bonded to the polyester, their durability is substantially increased.

Finishes & Lubricants

Ranges of chemical finishes are available for spinning, weaving, knitting, and braiding productivity, as well as functional properties. They combine low fiber to metal frictional properties, good inter-fiber cohesion, and excellent anti-static properties to maximize fiber, filament or yarn performance. For example 16-20% Lurol NF-782 aqueous emulsion spin finish is recommended for fine denier filament yarns such as polyester with 0.8-1.2% take up on the weight of the yarn. The emulsion is prepared by adding the finish slowly into rapidly agitating 45-50° C. water. The emulsion should be translucent; opalescent in concentrations up to 20%. Typical properties include a clear yellow appearance of the liquid at 25° C., gardner color <1, Viscosity cSt 56 and pH of 8.2 in 5% aqueous solution. It begins to freeze if stored below 10° C. If frozen, the product should be warmed above 25° C. and stirred before use to insure homogeneity. Some bactericide should be added to the emulsion to assure adequate storage life.

Finish level can be measured by conventional solvent extraction techniques, e.g., using a blend of isopropanol and n-hexane as solvents for polyester.

The spinning finishes according to the invention may contain emulsifiers, wetting agents and/or antistatic agents and, optionally, standard auxiliaries, such as pH regulators, filament compacting agents, bactericides, and conductive polymers. Suitable emulsifiers, wetting agents and/or antistatic agents are anionic, cationic and/or nonionic surfactants, such as mono- and/or diglycerides, for example glycerol, mono- and/or dioleate, alkoxylated, preferably ethoxylated and/or propoxylated, fats, oils, fatty alcohols, castor oil containing 25 mol ethylene oxide (EO) and/or 16-18 fatty alcohol containing 8 mol propylene oxide and 6 mol EO, if desired alkoxylated 8-24 fatty acid mono- and/or diethanolamides, for example optionally ethoxylated oleic acid mono- and/or diethanolamide, tallow fatty acid mono- and/or diethanolamide and/or coconut oil fatty mono- and/or diethanolamide, alkali metal and/or ammonium salts of alkoxylated, preferably ethoxylated and/or propoxylated, optionally end-capped 8-22 alkyl and/or 8-22 alkylene alcohol sulfonates, reaction products of optionally alkoxylated 8-22 alkyl alcohols with phosphorus pentoxide or phosphorus oxychloride in the form of their alkali metal, ammonium and/or amine salts, for examples phosphoric acid esters of ethoxylated 12-14 fatty alcohols, neutralized with alkanolmine, alkali metal and/or ammonium salts of 8-22 alkyl sulfosuccinates, for example sodium dioctyl sulfosuccinate and/or amine oxide, for example dimethyl dodecyl amine oxide. In considering this list of examples, it is important to bear in mind that many of the substances mentioned can perform not just one function, but several functions. Thus, an antistatic agent may also act as an emulsifier.

Suitable filament compacting agents are the polyacrylates, fatty acid sarcosides and/or copolymers with makeic anhydride known from the prior art (Melliand Textilberichte (1977), page 197), polyurethanes according to DE-A-38 30 468, pH regulators (e.g., C₁₋₄ carboxylic acids and/or C₁₋₄ hydroxycarboxylic acids (e.g., acetic acid and/or glycolic acid)), alkali metal hydroxides (e.g., potassium hydroxide), amines (e.g., triethanolamine), and bactericides.

UV Inhibitors

Ranges of commercially available high performance fibers are susceptible to ultra violet (UV) exposure. A list of typical stabilizers against UW, both radical formation & biodegradation, includes: 2-hydroxybenzophenones, 2-hydroxypenyl-2-(2H)-benzotriazoles, cinnamates, and mixtures thereof. These chemicals are capable of absorbing and dissipating UW energy, thus inhibiting UW degradation. Free radicals are neutralized by hindered amine light stabilizers (HALS), butylated hydroxyanisole (BHA) and butylated hydroxytoluene (BHT).

Antimicrobials

Antimicrobials include silver nitrate, iodized radicals (e.g., TRIOSYN®; Hydro Biotech), benzylalkonium chloride and alkylpyridinium bromide (cetrimide) or alkyltrimethylammonium bromide. It is within the scope of this disclosure to coat or impregnate the bifunctionalized polyester material disclosed herein as well as implants and prosthetic devices made therefrom with one or more materials which enhance its functionality, e.g., surgically useful substances, such as those which accelerate or beneficially modify the healing process when the material is implanted within a living organism. Thus, for example, antimicrobial agents such as broad spectrum antibiotics (gentamicin sulphate, erythromycin, or derivatized glycopeptides), which are slowly released into the tissue, can be incorporated to aid in combating clinical and subclinical infections in a surgical or trauma wound site. Other antimicrobials that can be used in the compositions of the invention include those described in U.S. Pat. Nos. 6,013,106; 6,464,971; 6,465,429; 6,471,974; 6,472,384; 6,472,424; 6,475,771; 6,479,454; 6,485,928; 6,492,328; 6,500,861; 6,506,737; 6,509,349; 6,436,445; 6,426,369; 6,423,748; 6,420,116; 6,417,217; 6,407,288; 6,387,928; and 6,376,670.

Growth Factors

To promote wound repair and/or tissue growth one or several substances can be introduced into the present composite biocompatible materials or impregnated into fabrics or prostheses made from the bifunctionalized polyester material. Exemplary substances include polypeptides such as human growth factors. The term “human growth factor” or “HGF” embraces those materials, known in the literature, which are referred to as such and includes their biologically-active, closely related derivatives. The HGFs can be derived from naturally occurring sources and are preferably produced by recombinant DNA techniques. Specifically, any of the HGFs which are mitogenically-active and as such effective in stimulating, accelerating, potentiating or otherwise enhancing the wound healing process are useful herein. Growth factors contemplated for use include HEGF (urogastrone), TGF-beta, IGF, PDGF, and FGF. These growth factors, methods by which they can be obtained and methods and compositions featuring their use to enhance wound healing are variously disclosed in U.S. Pat. Nos. 3,883,497; 3,917,824; 3,948,875; 4,338,397; 4,418,691; 4,528,186, 4,621,052; 4,743,679 and 4,717,717; European Patent Applications 0 046 039; 0 128 733; 0 131 868; 0 136 490; 0 147 178; 0 150 572; 0 177 915 and 0 267 015; PCT International Applications WO 83/04030; WO 85/00369; WO 85/01284 and WO 86/02271, and UK Patent Applications GB 2 092 155 A; 2,162,851 A, and GB 2 172 890 A, all of which are incorporated by reference herein.

The surface of the bifunctionalized polyester polymer of the invention can be modified with biologically-active proteins in order to emulate some of the natural properties of native vessels, thereby improving graft patency and healing. For example, the enzymatic, chemotactic, and mitogenic properties of thrombin can be inhibited by surface bound rHir (recombinant hirudin). This inhibition can significantly reduce blood product formation and maintain anastomotic smooth muscle cells in the quiescent state, thereby preventing the formation of anastomotic intimal hyperplasis. rhir has been shown to have potent anti-thrombin activity when covalently immobilized onto a DACRON® surface (see, e.g., Phaneuf M. D., et al., Biomaterials 18(10):755 (1997) and Berceli S. A., et al., J. Vasc. Surg. 27:1117 (1998)), or to another biomolecule (see, e.g., Phaneuf M. D., et al., Thromb. Haemostas. 71(4):481 (1994)). In addition, covalent linkage of VEGF (vascular endothelial growth factor) may permit complete endothelialization of the graft surface by both trans-anastomotic and trans-membrane (through the remaining porosity) cellular migration. Techniques for binding growth promoting factors to biocompatible materials are described in U.S. Ser. No. 09/139,507 entitled “Growth-Promoting Biocompatible Substances and Methods of Use Thereof,” and in Kubaska S. M. III, et al., Surgical Forum 49:322 (1998), which are herein incorporated by reference. Furthermore, as is discussed in detail below, the attachment of activated protein C (APC) to the surface of a medical device promotes a substantial reduction in thrombogenicity, while also improving endothelialization of the device.

Covalent linkage of protein to a biomaterial surface in order to create a “basecoat” layer has numerous beneficial advantages. Non-specific or covalent attachment of a protein coating can “passivate” a surface that is relatively thrombogenic, thereby decreasing adhesion of blood products such as platelets, red blood cells, and fibrinogen (Rumisek J., et al., Surgery 105:654 (1989)). Proteins incorporated as a basecoat layer can be used as a “scaffolding” in order to promote a specific response such as linkage of RGD peptides to promote cell adhesion (Lin H. B., et al. J. Biomed. Mater. Res. 345:170 (1994)). Additionally, increasing the angstrom distance between a biologically-active molecule and the surface via polyethylene oxide groups can reduce steric hindrance on the target molecule, thereby maintaining activity (Park K. D., et al., J. Biomed. Mater. Res. 22:977 (1988)).

Covalent linkage of a protein “basecoat” layer can serve as the spacer between rHir/VEGF and the biomaterial surface. Albumin can be used as the basecoat moiety. Albumin, which is in natural abundance in circulating blood, has numerous beneficial results in vitro and in vivo (see, e.g., Kotteke-Marchant K., et al., Biomaterials 10:147 (1989) and Phaneuf M. D., et al., J. Applied Biomater. 6:289 (1995)). Utilization of a basecoat layer permits significant amplification of potential binding sites for secondary protein attachment via heterobifunctional crosslinkers; thus creating a biomaterial surface with distinct properties for a specific application. This technique has been used to provide numerous binding sites for rhir, for example (see, e.g., Phaneuf M. D., et al. ASAIO J. 44:M653 (1998) and Phaneuf M. D., et al., Biomaterials 18(10):755 (1997)). Examples of other basecoat proteins include, but are not limited to, collagen and fibronectin. Alternatively, the basecoat may be synthetic, such as, e.g., a Lys-Tyr moiety or polyethylene oxide.

Additives/Modifiers

Additional modifiers that can be applied to the bifunctionalized polyester material include, but are not limited to, the following: thermally conductive agents (e.g., graphite, boron nitride), ultraviolet-absorbing agents (e.g., benzoxazole, titanium dioxide, zinc oxide, benzophenone and its derivatives), water repellent agents (e.g., alkylsilane, stearic acid salts), therapeutic agents (e.g., antibiotics, hormones, growth factors), stain resistant agents (e.g., mesitol, CB-130), rot resistant agents (e.g., zinc chloride), adhesive agents (e.g., epoxy-resin, neoprene), anti-static agents (e.g., amines, amides, quaternary ammonium salts), biocidal agents (e.g., halogens, antibiotics, phenyl mercuric acetate), blood repellents (e.g., monoaldehyde urea resin), dye and pigments, electrically conductive agents (e.g., metal particles, zinc oxide, stannic oxide, indium oxide, carbon black, silver, nickel), electromagnetic shielding agents (e.g., hypophosphorous, carbon-phenol resin compounds), and flame retardant agents (e.g., aluminum hydroxide, borax, polyamide, magnesium hydroxide, polypropylene).

### EXAMPLE 1

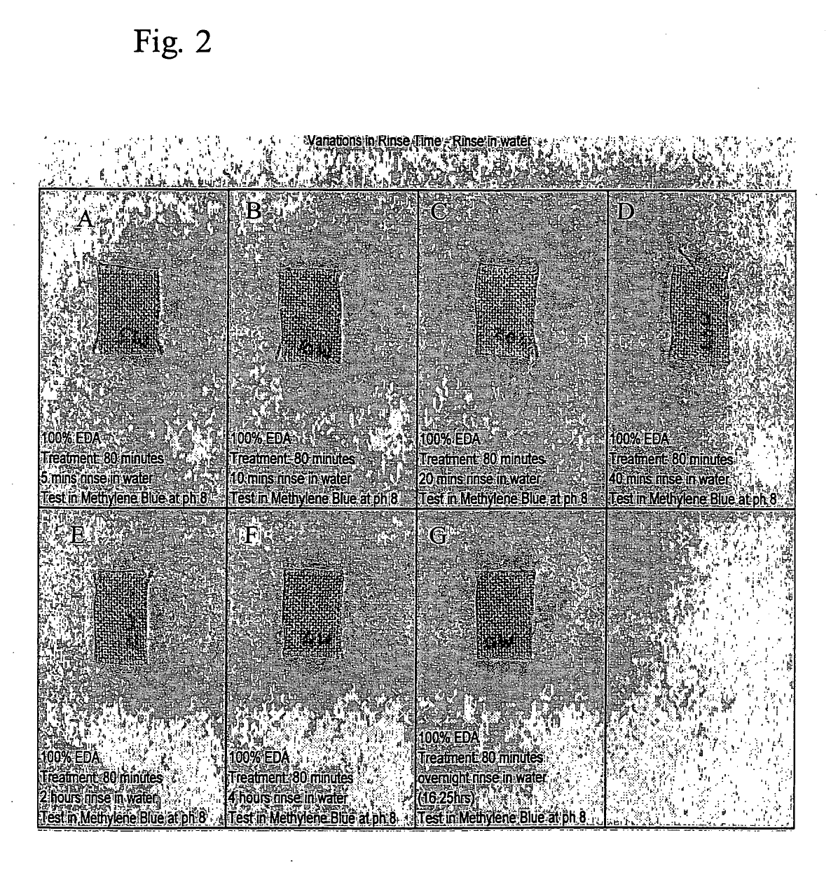

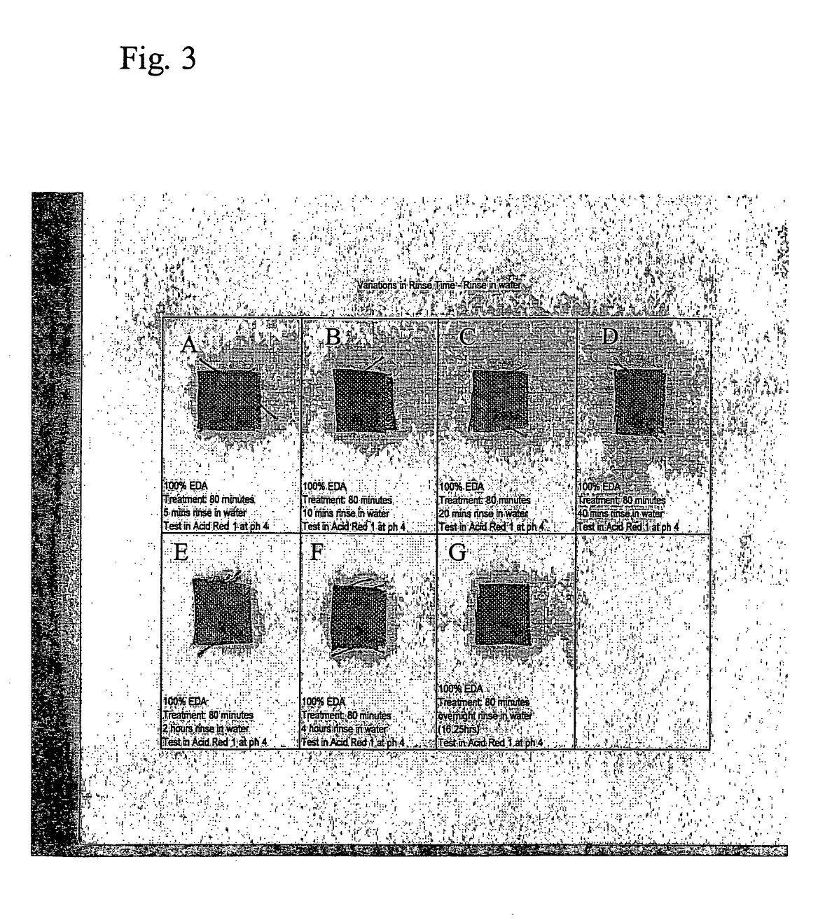

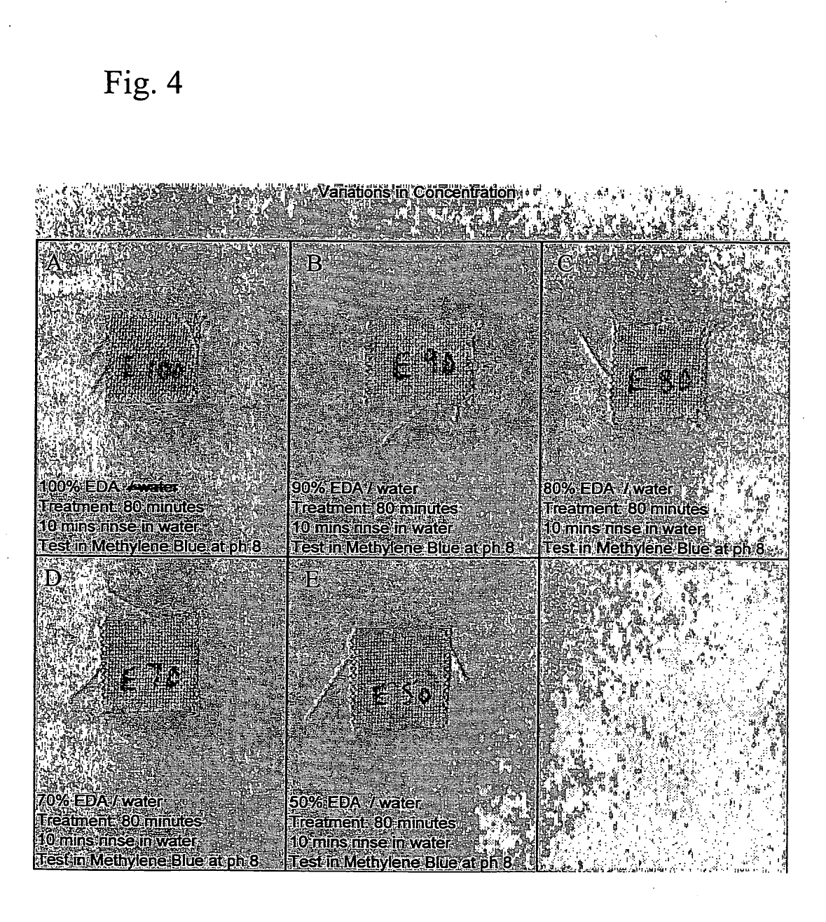

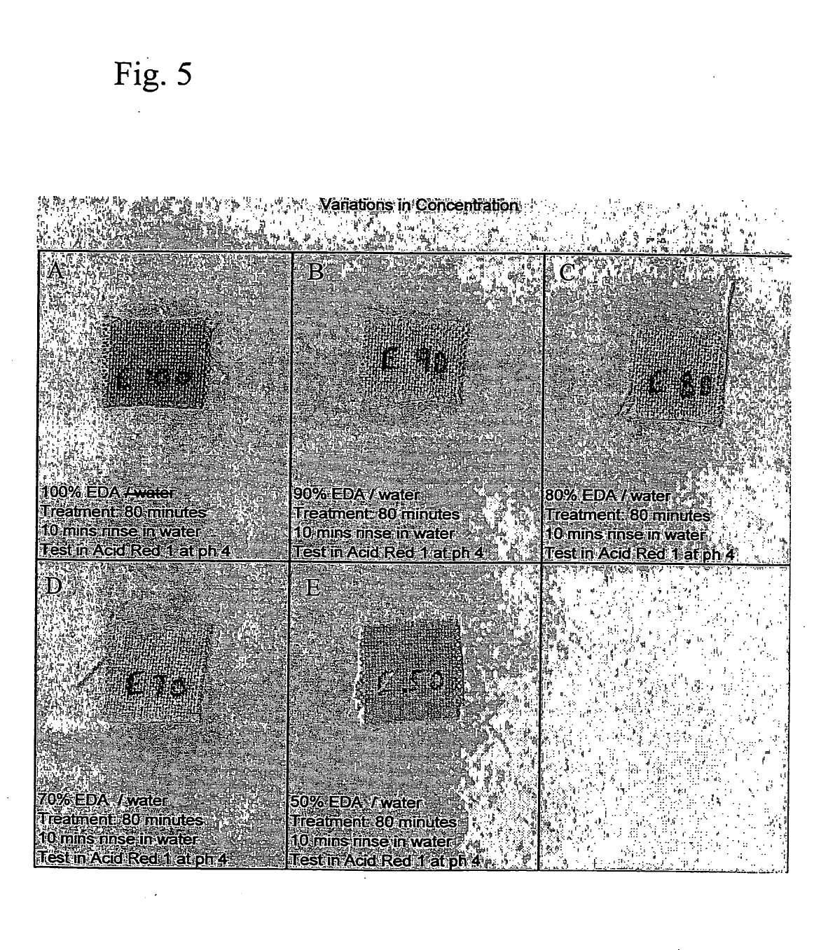

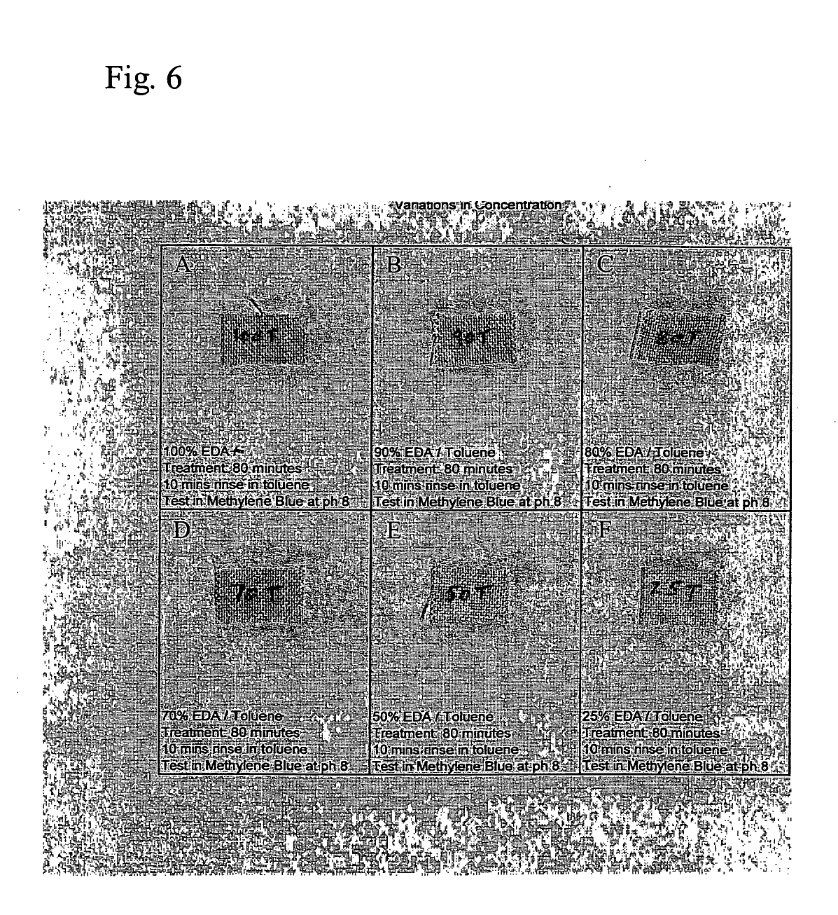

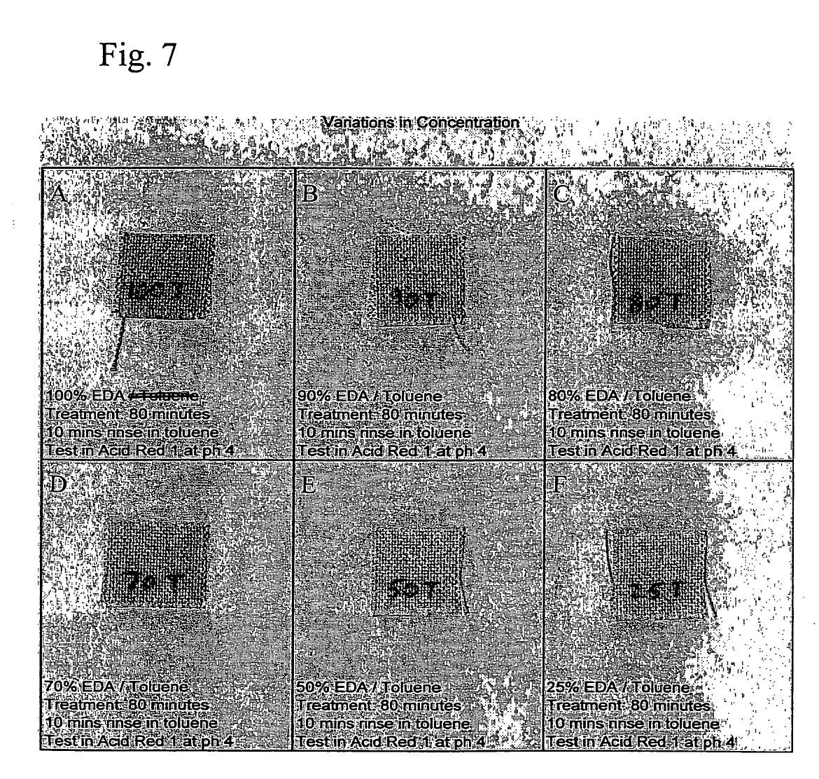

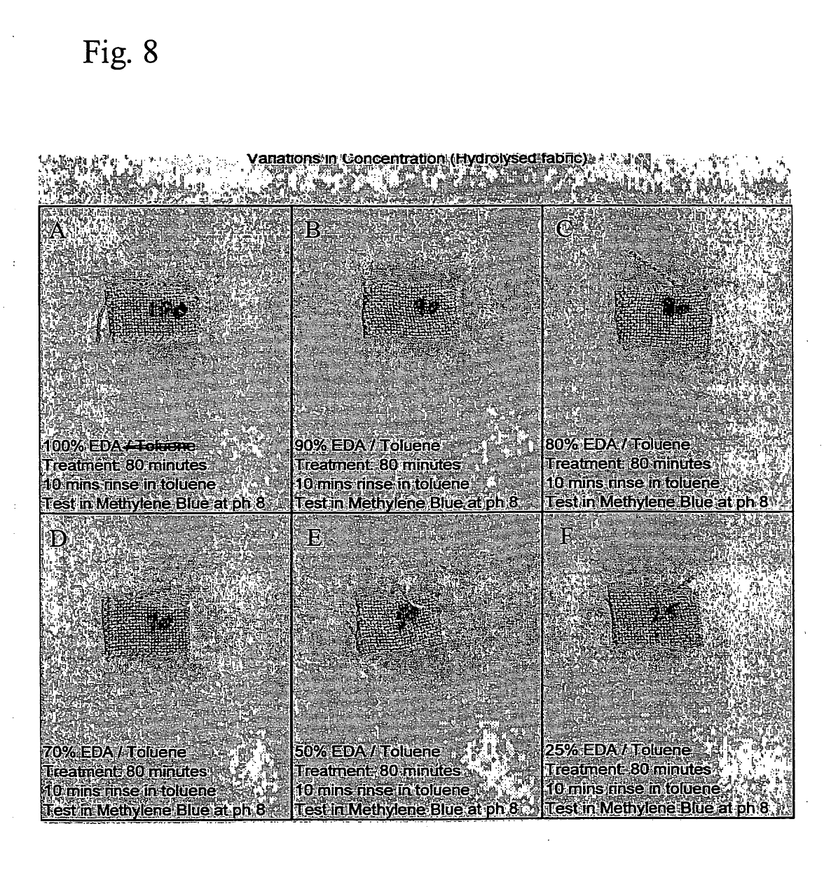

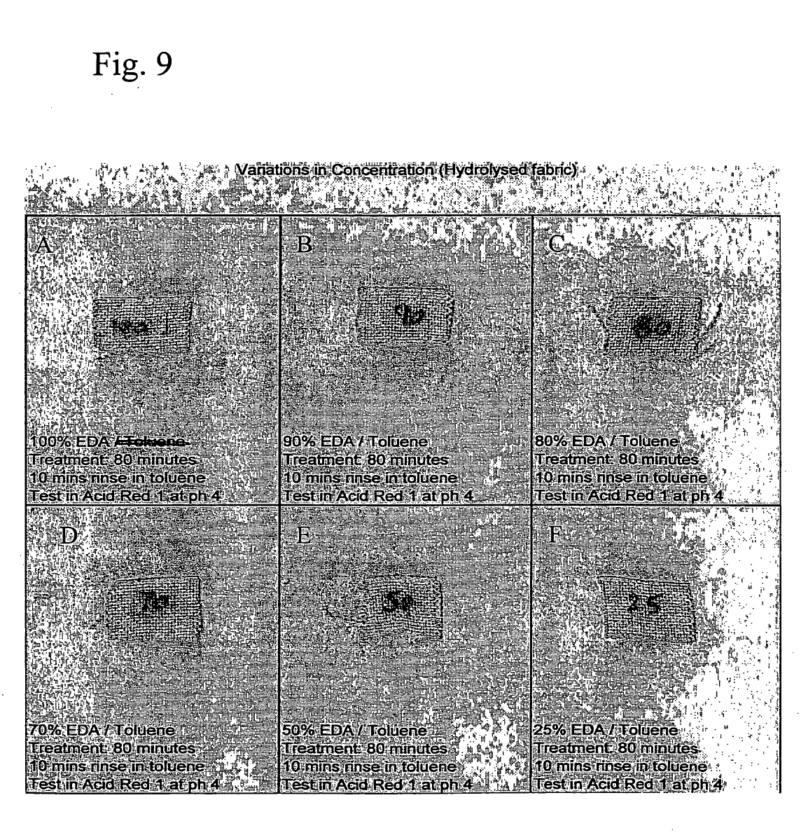

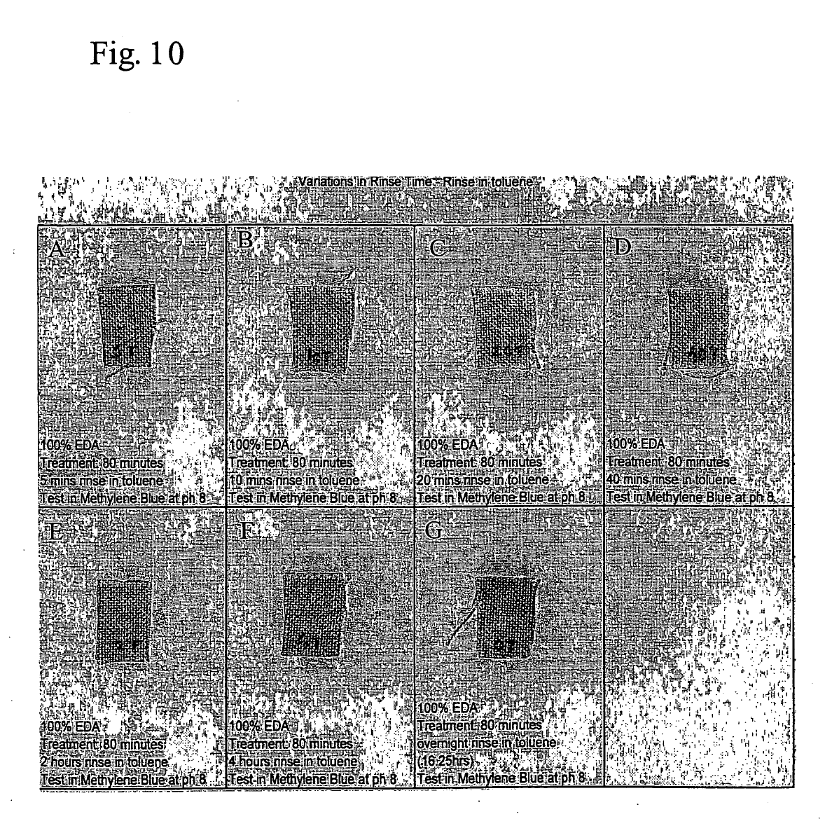

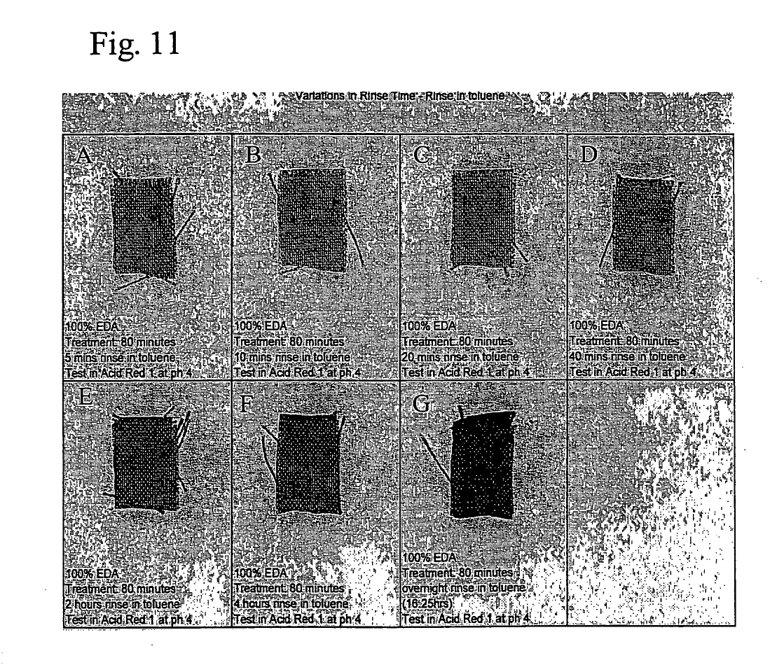

We tested whether we could generate amine functional groups on the surface of polyester by treatment with ethylene diamine (EDA). Exposure of the polyester to EDA created both carboxylic and amine groups within the polymer structure as evidenced by uptake of both methylene blue (FIGS. 2A-2G) and acid red (FIGS. 32A-3G). Formation of these groups could also be regulated by EDA concentration but was not significantly altered by the rinse time (see FIGS. 4A-4E for methylene blue determination of carboxylic acid groups and see FIGS. 5A-5E for acid red determination of amine groups). For the hydrolyzed material (HYD), carboxylic acid content decreased with increasing EDA concentration whereas amine content increased, suggesting amine groups were limited to the outer periphery of the fiber. Amine content in the hydrolyzed segments was not as elevated as the scoured segments (CNTRL). For the CNTRL and HYD polyesters, employing toluene as the solvent at lower concentrations increased carboxylic acid (see FIGS. 6A-6F for CNTRL and FIGS. 8A-8F for HYD) and amine (see FIGS. 7A-7F for CNTRL and FIGS. 9A-9F for HYD) formation. In contrast to the water washing studies, exposing the segments to a prolonged toluene rinse increased formation of both carboxylic acid (FIGS. 10A-10G) and amine (FIGS. 11A-11G) functional groups.

Determination of Amine/Carboxylic Acid Content via Textile Dye Uptake

EDA exposure to scoured (CNTRL) and hydrolyzed (HYD) segments resulted in a yellowish-brown coloration as compared to unmodified CNTRL and HYD surfaces, both of which remained white. Using acid red uptake, CNTRL-EDA (0.82±0.10 nmoles/mg) and HYD-EDA (0.32±0.02 nmoles/mg segments had 43- and 8-fold greater amine content as compared to their respective controls. Amine formation was 2.6-fold greater using CNTRL as compared to HYD material. Using methylene blue uptake, carboxylic acid content in the CNTRL-EDA segments increased 18-fold whereas a 40-fold decrease in carboxylic acid content occurred for the HYD-EDA segments. This carboxylic acid group loss in the HYD-EDA segments may be due to EDA reaction with the carboxylic acid groups created during the initial alkaline hydrolysis.

Determination of Primary Amine Functional Groups via Sulfo-SDTB

Sulfo-SDTB analysis of the control and EDA treated materials confirmed the creation of amine groups into the polymer structure of the EDA treated DACRON®. Additionally, amine content in the CNTRL-EDA (1.06±0.11 nmoles/mg) and HYD-EDA (0.36±0.03 nmoles/mg) segments was comparable to the results obtained in the dye uptake study.

Physical Characteristics of the EDA-Modified Polyester

Fiber weight loss from the CNTRL-EDA (2.3±0.55%) and HYD-EDA (1.3±0.25%) segments was 3.8- and 2.0-fold greater than their respective controls. The difference in fiber weight loss between HYD-EDA and CNTRL-EDA segments (HYD-EDA segments lost 1.9-fold less fibers than the CNTRL-EDA segments) could again be attributed due to EDA reaction with the carboxylic acid groups previously created on the fiber surface via alkaline hydrolysis thus restricting deep EDA penetration into the fiber.

Tensile strength of the CNTRL-EDA and HYD-EDA segments was decreased 1.7 and 1.3 fold as compared to CNTRL and HYD segments, respectively. Ultimate elongation also followed a similar trend, with a 1.6 and 1.3 fold loss in elongation in the CNTRL-EDA/HYD-EDA segments. Comparable to the fiber weight loss study, HYD segments were less affected by exposure to EDA as compared to the CNTRL segments.

Accessible amine and carboxylic acid groups have been created within the polymer backbone of both CNTRL and HYD polyester (e.g., DACRON®) materials as determined by dye uptake and sulfo-SDTB indicator. Additionally, the bulk physical characteristics of both materials still remain.

Materials and Methods

Polyester Preparation: Segments (5 cm×5 cm) were cut from a large woven fabric sample and washed in 500 ml scouring solution (10 g Na₂CO₃, 10 ml Tween 20 in 1 L double distilled water (ddH₂O)) for 30 minutes at 60° C. Samples were then rinsed in 500 ml ddH₂O for 30 minutes at 60° C. (CNTRL) and air-dried overnight. Some of these scoured segments were then exposed to 500 ml of 0.5% NaOH at 100° C. for 30 minutes. Alternatively, other NaOH conditions ranging from 1-20% could also be employed. These pieces were then rinsed with ddH₂O (room temperature) and air-dried overnight at room temperature.

Formation of Amine and Carboxylic Acid Groups: The primary procedure employed for this study was to incubate a 5 cm×5 cm segment of either control or hydrolyzed polyester into 100% ethylene diamine (EDA, Sigma) for 80 minutes at room temperature. The segments were then removed and placed into distilled water overnight (˜16 hours) at room temperature, followed by air-drying at 60° C. for 2 hours. Several other approaches were performed. EDA concentration, rinse times and solvent type were performed for both control and hydrolyzed DACRON®.

Determination of Amine and Carboxylic Acid Content: Methylene blue, a cationic dye, was employed to qualitatively determine carboxylic acid groups within the EDA-exposed polyester segments. Briefly, a 500 ml stock solution (500μg/ml) of methylene blue was prepared (80% Purity) in 0.1 M Tris-CL pH 8.0. A working solution of methylene blue was prepared by aliquotting 1 ml of the stock solution and bringing to a total volume of 100 ml with Tris buffer (5 μg/ml). Segments (1 cm²) were then cut from scoured and hydrolyzed EDA segments. Working MB solution (4-10 ml) was added to each segment, and incubated for 1 hour on an inversion mixer. The segments were removed and placed into wash solution consisting of Tris buffer for one hour. Pre and post dye bath solutions were read at 611 nm using Tris buffer as blank. Segments were then grossly observed for color uptake and shade differences and photographed. Carboxylic acid content (nmoles/segment) weight (mg) was calculated using standard textile equations.

For amine content, acid red 1 (ARI), an anionic dye, was employed to quantitatively and qualitatively assess total (primary and secondary) amine content in the DACRON®-EDA segments. Briefly, a 500 ml stock solution of ARI (0.5 mg/ml, dye purity=60%) was prepared in 0.01 M MES pH 4.5 (MES). A working solution of ARI was prepared by aliquotting 10 ml of the stock solution and bringing to a total volume of 100 ml with MES buffer (50 mg/l). Segments (1.0 cm²) were cut from the respective treatments. Working ARI solution (2-3 ml) was added to each segment and incubated for 1 hour on an inversion mixer. The segments were removed and placed into wash solution of MES buffer for one hour. Pre and post dye bath solutions were read at 530 nm using MES buffer as the blank. Segments were then grossly observed for color uptake and shade differences and photographed. Amine content (nmoles)/segment weight (mg) was calculated using standard textile equations.

Determination of Primary Amine Functional Groups via Sulfo-SDTB: A stock buffer consisting of 50 mM sodium bicarbonate, pH 8.5 was prepared. CNTRL, HYD, CNTRL-EDA, and HYD-EDA segments (n=4/test condition; 1.0 cm²) were cut and weighed. Sulfo-SDTB (3 mg) was weighed and dissolved in 1 ml dimethyl formamide. After thorough mixing, the sulfo-SDTB solution was brought up to a total volume of 50 ml with the stock sodium bicarbonate buffer (working sulfo-SDTB solution). Stock buffer (1 ml) and 1 ml working sulfo-SDTB solution were added to each tube and reacted for 40 minutes at room temperature on an orbital shaker. Segments were then removed and washed for 10 minutes in 5 ml of distilled water on an inversion mixer. Immediately following the wash, 2 ml of a perchloric acid solution was added to each segment. Segments were reacted for 15 minutes on the inversion mixer. The reaction solution (1 ml) was then removed and absorbance at 498 nm was measured. Using the extinction coefficient for sulfo-SDTB and the segment weight, amine content (nmoles/segment weight (mg) was determined.

Physical Characterization of EDA-Modified DACRON®: Fiber weight loss was determined post-exposure to either distilled water (control) or EDA. CNTRL and HYD segments were prepared as previously described. Segments (4 cm²) were cut from each segment type (n=8 segments/treatment) and weighed. Half of the segments for each treatment were placed into distilled water and the other half placed into 100% EDA for 80 minutes. All segments were then transferred to distilled water for 16 hours, followed by air-drying at 60° C. for 2 hours. Segments were then reweighed, with the difference in segment weight determined.

Tensile strength and ultimate elongation were then determined. CNTRL, HYD, CNTRL-EDA, and HYD-EDA segments (width=1 inch, length=2 inches) were cut. A Q-test apparatus was calibrated at the time of use under a controlled climate (room temperature—24.7° C., humidity—75%). A 100-pound load cell was used and a pull rate of 12 inches/minute was set. A gauge length of 0.75 inch was set into the apparatus, with a total of 1.25 inches of each segment placed into the clamps. Stretching was then initiated and automatically stopped at the break of each segment. The peak load at break (1 lb) and the ultimate elongation for each segment was determined.

### EXAMPLE 2

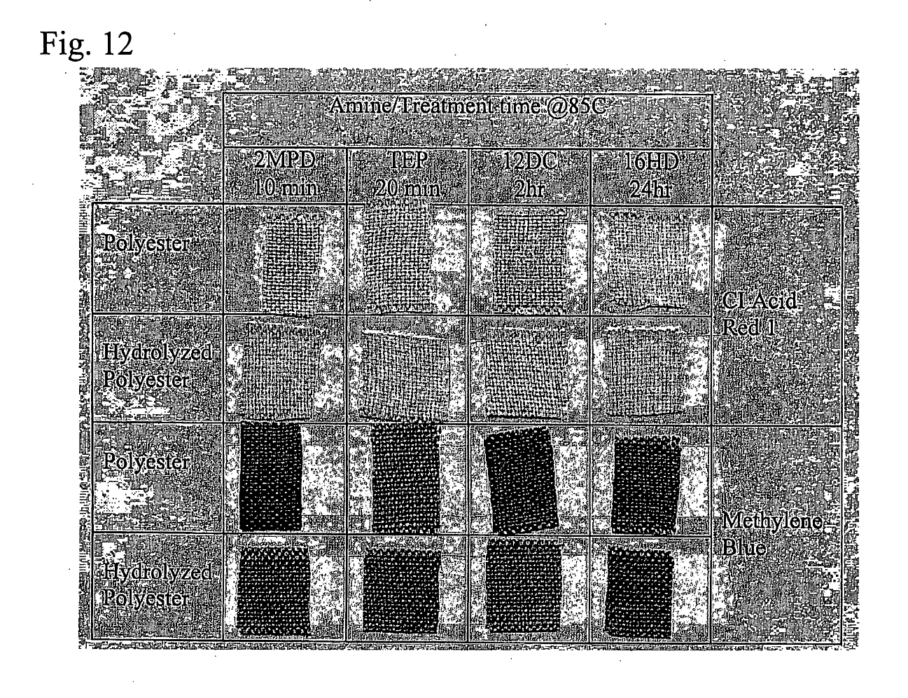

We tested whether treatment of polyester fabric with amines other than EDA would result in the generation of functional amine groups. Polyester and hydrolyzed polyester were treated with four different multifunctional amines at a range of times and temperatures, and then dyed in diagnostic dyes. We specifically tested the uptake of CI Acid Red 1 or Methylene Blue dye by polyester fabric (hydrolyzed or unhydrolyzed) after treatment of the fabric at 85° C. with 2-methylpentamethylene diamine (2 MPD) for 10 minutes, tetraethylenepentamine (TEP) for 20 minutes, 1,2-diaminocyclohexane (12 DC) for 2 hours, and 1,6-hexanediamine (16 HD) for 24 hours. The results of these treatments are shown in FIG. 12. The loss in tensile strength caused by these treatments is shown in Table 1.

TABLE 1
Effect of diamines on fabric strength

| | Amine/Treatment time @85 C. | | |
|---|---|---|---|
| | 2MPD 10 min | TEP 20 min | 12DC 2 hr |
| | % strength loss | | |
| Polyester | 15 | 15 | 64 |
| Hydrolyzed | 0 | 0 | 20 |
| Polyester | | | |

While these amines differ in the ease of reaction with polyester (roughly similar effects occurring at times ranging from 10 minutes to 24 hours), they are effective at providing amine groups at the fiber surface. It is notable that they also hydrolyze the surface and yield carboxylic acid groups (see below).

Treatment with EDA in Toluene

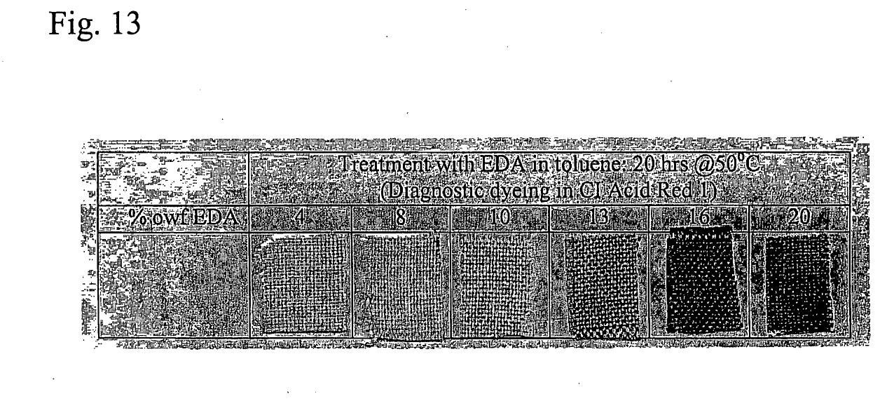

The effect of increasing EDA concentration when applied at constant temperature and time (i.e., 20 hours at 50° C.) is shown in FIG. 13. As expected, increasing the applied concentration of EDA results in the formation of more amine groups on the fiber, as is determined by the uptake of C.I. Acid Red 1 dye, and the reaction seems thus to be readily controllable in the manner of dye application.

Treatment with EDA

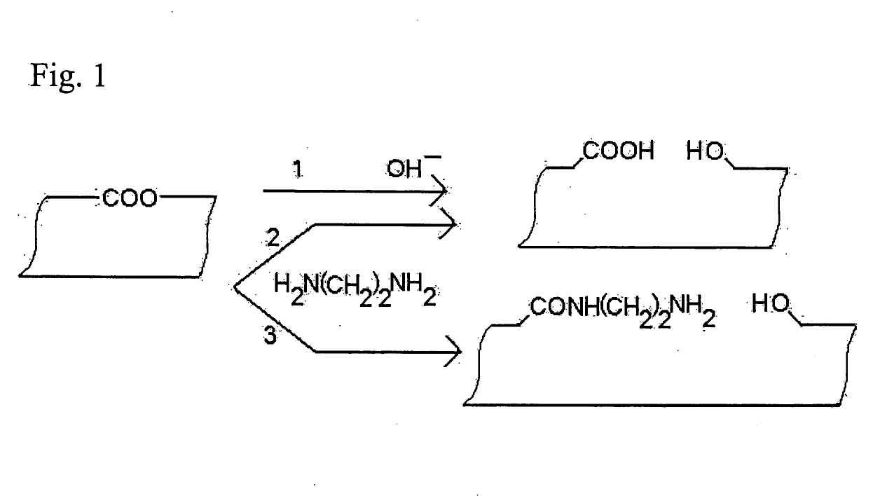

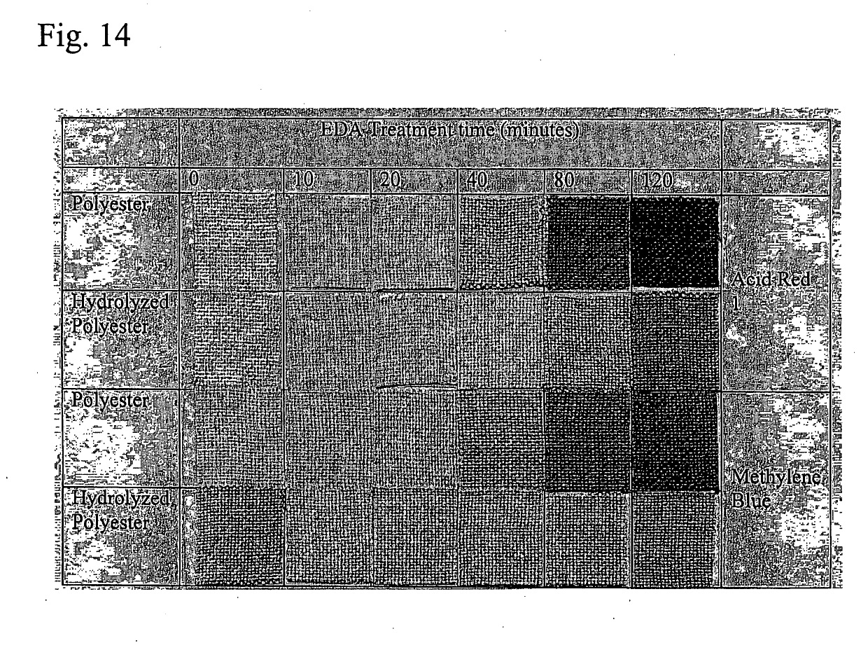

For the sake of simplicity, the basic reaction of polyester (original and hydrolyzed) in pure EDA at room temperature was used for the majority of the study to determine its effect on the fiber and its properties. The extent of treatment was controlled by the time of immersion in EDA. The gradual incorporation of amine groups at the polyester surface was followed by dyeing with C.I. Acid Red 1. The presumed reaction scheme is shown in FIG. 1, reaction 3. FIG. 14 shows the darkening of shade as treatment time increases. Noticeable is that the treatment on hydrolyzed material is less effective at generating amine groups. Perhaps more surprising is the result of diagnostic dyeing in Methylene Blue. As expected, the hydrolyzed material has carboxylic acid groups present, but the treatment of unhydrolyzed material with EDA also generates carboxylic acid groups. This is presumably due to hydrolysis involving the strongly basic diamine and any small amount of water present (FIG. 1, reaction 2). On treatment with EDA, the number of carboxylic acid groups in the hydrolyzed material initially decreases, and then increases again.

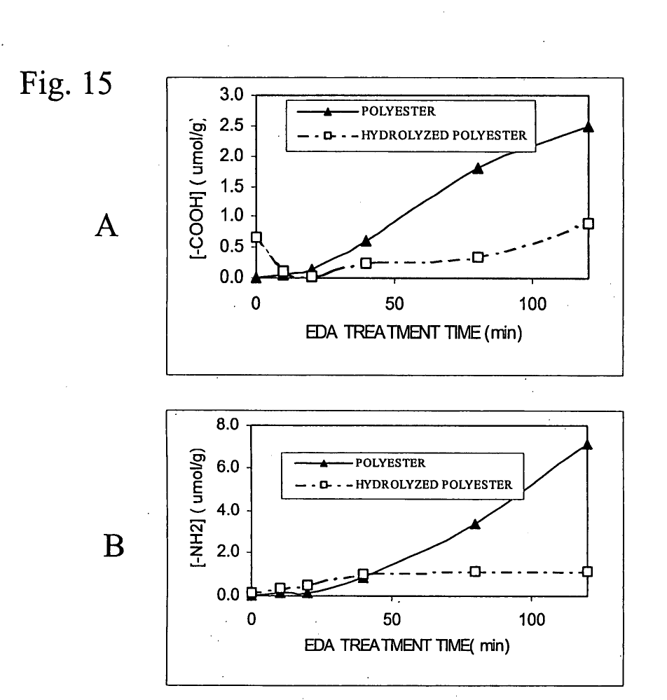

The quantification of these functional groups via dyeing with dyes of known purity produced the results shown in FIGS. 15A and 15B. Again, the generation of fewer amine groups and the initial loss of carboxylic acid groups on the hydrolyzed material is shown.

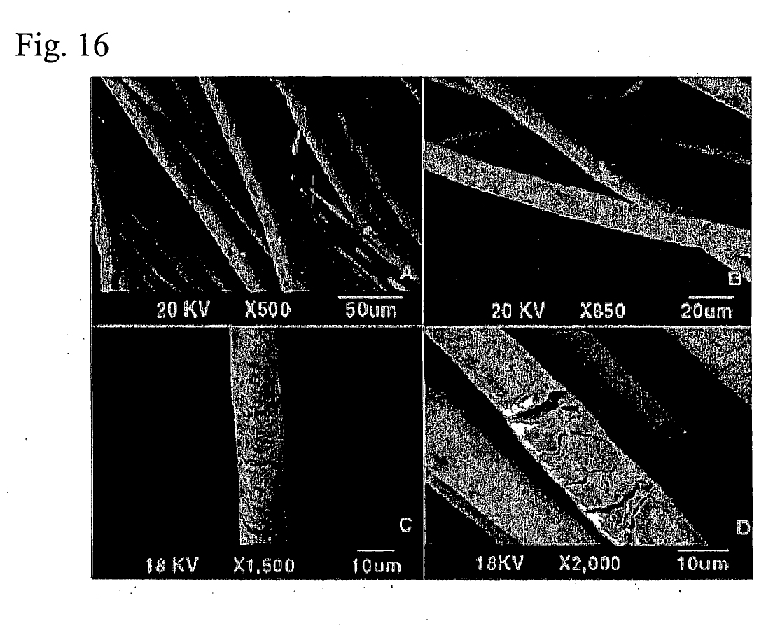

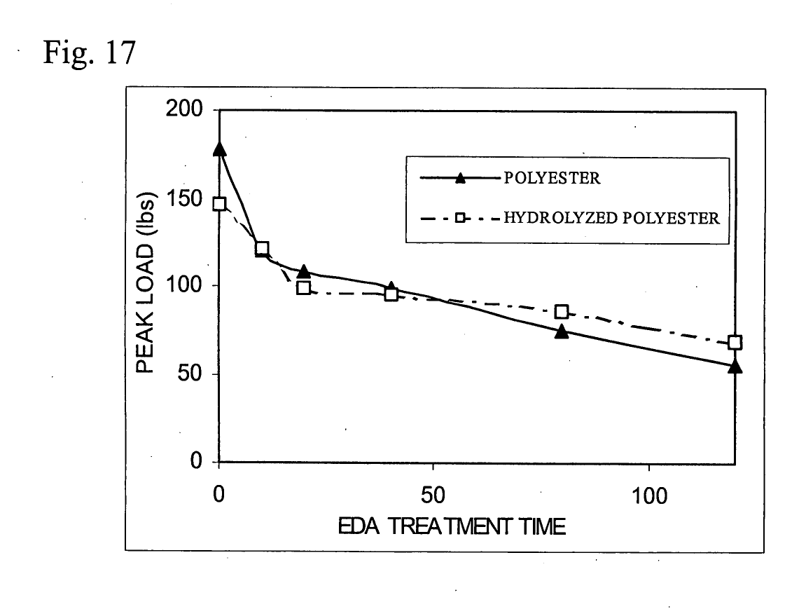

An electron micrograph of untreated polyester and hydrolyzed polyester, and both after treatment with EDA is shown in FIGS. 16A-16D. It seems apparent that aminolysis, as found in previous studies, is a more penetrating treatment for polyester. Examination of these images suggests that the aminolysis, once started, proceeds more quickly in the initial areas of attack: areas of cracking are isolated among apparently undamaged material. Avny and Rebenfeld postulated an “induction” period and a subsequent “autoaccelerated” reaction (J. Applied Polymer Science 32:4009, 1986). Cracking is also visible on extensively treated fibers using visible microscopy, and cross-sections of the diagnostically dyed fibers show the treatment to be confined to the fiber surface. The slowing of aminolysis by a previous hydrolysis is also apparent when the effect of this treatment on the tensile strength of the material is considered. FIG. 17 shows the change in tensile strength with time of EDA treatment. After an initial drop (comparable with the loss of strength on hydrolysis alone) the tensile strength falls more slowly as the treatment continues. This again seems to suggest that the effect is happening more quickly on a few areas, rather than very generally. Weight loss data supported this contention (Table 3): only after comparatively long treatment times is a significant weight loss noted.

TABLE 3
Weight Loss on EDA Treatment

| | EDA Treatment | Weight loss |
|---|---|---|
| | Time (min) | (%) |
| Polyester | 80 | 0.85 |
| | 120 | 3.17 |
| Hydrolyzed | 80 | 0.15 |
| Polyester | 120 | 0.33 |

Efforts to determine the absorbency (wetting time) and static properties of these EDA-treated material were unsuccessful: all materials gave results that varied widely. The Soil Release ratings did, however, indicate that the EDA treatment has an effect (Table 4).

TABLE 4
Soil Release Ratings of Treated Polyester

| | SR Rating |
|---|---|
| Untreated | 3.63 |
| Hydrolyzed | 4.37 |
| EDA-treated | 4.13 |
| Hydrolyzed/EDA treated | 4.94 |

All treatments produced an improvement over untreated polyester. Alkaline hydrolysis, as previously established, gives an improvement in soil release. EDA treatment alone was less beneficial. A combination of hydrolysis and EDA treatment, however, gave excellent soil release properties.

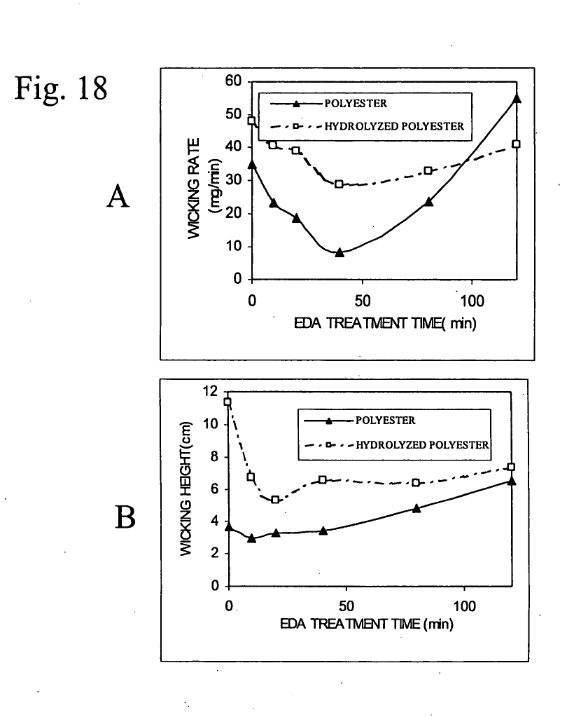

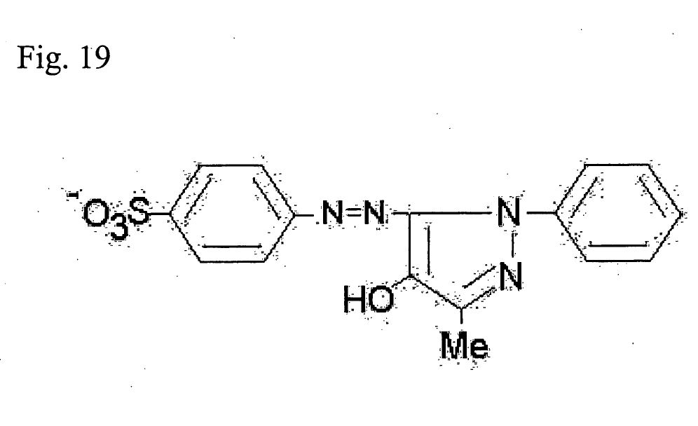

The greater hydrophilicity of surface that the increased soil release properties imply is also represented in the wicking data (FIG. 18). The hydrolyzed surface wicks water at a greater rate when distance is measured. However, when the weight of water is considered, at longer treatment times the EDA treated material takes up a slightly greater weight of water; this is possibly due to the greater access to the fiber interior allowed by the surface cracking. In both cases there is a suggestion that at shorter times of EDA treatment, the wicking performance is reduced.

The treatment of polyester with bifunctional aliphatic amines, especially ethylene diamine, generates amine groups on the fiber surface, as expected. Somewhat surprisingly, the reaction results in the simultaneous formation of carboxylic acid groups in a manner akin to the familiar alkaline hydrolysis. The reaction is slower when applied to polyester that has previously been subjected to alklaline hydrolysis. The reaction is readily controllable, and when applied to untreated or hydrolysed polyester has the potential to provide polyester surfaces with varying levels of amine and carboxylic acid functionality. The treatment is of great potential use in modifying the biomedical properties of polyester, and will allow for the binding of different biologically-active agents (e.g. anti-thromboytic agents (e.g., anti-thrombin), thrombolytic agents, growth-promoting/inhibiting agents, antimicrobial or antifungal agents) to give multifunctional materials.

As would be expected from a more hydrophilic surface, wicking and soil release are improved by the treatment.

Materials and Methods

Polyester Material: A plain weave 100% polyester fabric was used in all experiments (Style 755, Testfabrics, Inc., West Pittston Pa.)

Chemicals: Chemicals used were laboratory grade, including ethylene diamine, (“EDA”,99%) and sodium hydroxide. All water used in the experiments was de-ionized. Methylene Blue was obtained in 99% purity. CI Acid Yellow 4, previously synthesized in our laboratory, was purified from salt impurities by extraction in N,N-dimethylformamide.

Apparatus: An Ahiba Polymat (Datacolor International) dyeing machine was used in the hydrolysis treatment: other reactions were carried out in simple glassware. A Cary 50 UV-Visible Spectrophotometer, Varian Pty Ltd, was used in measuring dye uptake. A Qtest CRE (Constant Rate of Extension) tester, MTS Systems Corporation was applied to determine the tensile property. A Joel 5900 Scanning Electronic Microscope was used to examine the morphological modification on the fiber surface.

Fabric Treatment: Polyester fabric was subjected to alkaline hydrolysis by treatment in 1.0% w/v sodium hydroxide for lhr at 98° C., LR 40:1, followed by rinsing in water and air-drying. These conditions were earlier found to provide surface carboxylic acid functionality with minimal strength and weight loss. Treatments in bifunctional amines were carried out under a range of conditions.

Multifunctional amines 1,6-hexanediaamine (16 HD), 2-methylpentamethylene diamine (2 MPD), 1,2-diaminocyclohexane (12 DC) and tetraethylenepentamine (TEP) were applied to polyester at 100% concentration (at 10:1 liquor ratio) in glass vials at a range of temperatures and times in a laboratory oven. Ethylene diamine was applied to untreated polyester from a range of solution concentrations in toluene. Specimens were rinsed in acetone and then water, and dried.

Both untreated and hydrolyzed polyester fabric specimens were treated by immersion flat in ethylene diamine (10:1 LR) at room temperature (25° C.) for a range of times. After each treatment, specimens were removed and rinsed in de-ionized water until the water reached neutral, and air dried. Fabrics were conditioned under standard conditions for 24 hours before physical tests.

Tests: The Following Tests Were Carried Out on Treated and Untreated Fabrics:

1. Surface functional groups were determined by dye uptake experiments. For simple visual analysis, carboxylic acid groups were visualized by the uptake of Methylene Blue from a 50:1 LR bath of 0.1 g/l solution in 1 g/l ammonia, with a treatment at 60° C. for 20 minutes. Amine groups were visualized by dyeing in C.I. Acid Red 1 (0.1 g/l in 1 g/l acetic acid, 50:1 LR, 60° C., 20 min).

For more accurate quantification, dyeing with Methylene Blue was carried out with 0.25% owf of dye, 50:1 liquor ratio, with temperature set to 40-50 ° C. for 50 minutes. Ammonium hydroxide was used to adjust the dye bath to pH 9.5. Amine groups were quantified by the uptake of purified CI Acid Yellow 4 under the same conditions, except that the pH was adjusted to 4.0 with acetic acid. This dye was chosen since it is of known formula and is monosulfonated (FIG. 11).

After dyeing, solutions were diluted to a convenient concentration and their absorbance measured. Dye uptake was calculated by reference to pre-established absorbance/concentration relationships.

The amino or carboxylic acid groups were quantified using the following equation:

Functional Group Density=(Q/M)/W

Where Q represents the amount of dye taken up, M is the molecular weight of the Dye, and W is the weight of the fabric.

2. Weight Change was determined by measuring the constant oven-dried weight of fabrics before and after aminolysis and is expressed in percentage. Specimens in this test were pre-raveled 1 cm from the edges to avoid weight loss caused by raveling of yarns from fabric during treatment and rinsing:

3. Tensile Loss was measured on 25×150 mm raveled fabric stripes obtained from the warp direction of conditioned fabric. Using a crosshead speed of 200 mm/min and gage of 75 mm, breaking load and elongation of specimens was determined. % Loss of tensile strength was calculated.

4. Wicking Properties were assessed in terms of both the rate and amount of water wicked. Specimens of 25×150 mm were weighted at one end and immersed to a depth of 10 mm in a beaker of water on an analytical balance (0.1 mg; FIG. 12). The weight of the beaker was recorded every minute over a ten-minute period, and the average rate of wicking (mg/min) calculated. In addition, the height to which the wicked water reached was measured, and the average height per five minutes calculated.

5. Wetting Time was measured using AATCC TM 79-2000 (Absorbency of bleached textiles).

6. Electrostatic Cling was measured using AATCC Method 115-2000.

7. Soil Release Properties were assessed using AATCC Test Method 130-2000.

### EXAMPLE 3

We next sought to determine whether the generation of carboxylic acid or amine functional groups on polyester could be used to provide potential individual “anchor” sites for covalent attachment of biologically-active proteins. To address this issue, we modified polyester (DACRON®) as is described herein and quantified the protein binding to the carboxylic acid and amine groups on the surface.

Woven DACRON® patches (1 cm²) were treated with EDA for 80 minutes at 25° C. Patches were divided into three groups: untreated DACRON® (CTRL), control-EDA (C-EDA) DACRON®, and Tr-EDA DACRON® (EDA-treated DACRON® reacted with Traut's Reagent, a heterobifunctional crosslinker that reacts with primary amine groups on the surface). Bovine serum albumin (BSA, 1 mg) was radiolabeled with ¹²⁵I. BSA was then reacted with the heterobifunctional crosslinker Sulfo-SMCC for 20 minutes at 37° C. Each group of patches was then incubated on an orbital shaker for 3 hours at 25° C. with ¹²⁵I-BSA-Sulfo-SMCC.

A second study involved CTRL and C-EDA segments as well as EDC-EDA segments (EDA-treated patches reacted with EDC, a carbodiimide crosslinker that reacts with carboxylic acid groups on the surface). BSA (1 mg) was radiolabeled with ¹²⁵I BSA. Each group of patches was then incubated with ¹²⁵I on an orbital shaker for 3 hours at 25° C.

A third study, which again involved CTRL and C-EDA patches, also assessed Tr-EDC-EDA patches (EDA-treated DACRON® patches reacted simultaneously with the Traut's Reagent and EDC). BSA (1 mg) was radiolabeled with either ¹²⁵I or ¹³¹I. The ¹²⁵I-BSA was again reacted with Sulfo-SMCC. Each group of patches was then simultaneously incubated with ¹²⁵I-BSA-Sulfo-SMCC and ¹³¹I-BSA on an orbital shaker for 3 hours at 25° C. For each study, patches were washed in detergent and sonicated, followed by gamma counting. Lowry and TCA assays were performed to assess BSA concentration and radiolabeling efficiency, respectively. Using this data, specific activity for the BSA samples was calculated. Protein binding was then determined as amount of protein (ng) per DACRON® segment weight (mg).

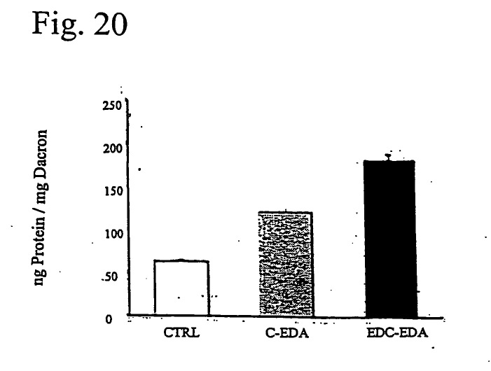

Albumin binding, which was either non-specific or covalent, occurred on all of our surfaces. For the first single binding study, ¹²⁵I-BSA binding to the Tr-EDA group (360±10 ng/mg) was 20.4 fold and 2.3 fold greater than CTRL (1.8±0.3 ng/mg, p=2.5×10⁻⁸) and C-EDA (155±3 ng/mg, p=8.8×10⁻⁷) segments, respectively (FIG. 20).

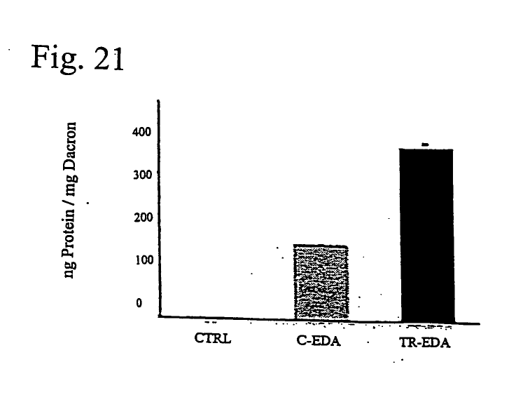

For the second single protein binding study, ¹²⁵I-BSA binding to the EDC-EDA segments (184±6 ng/mg) was 2.9 fold and 1.5 fold greater than CTRL (64±3 ng/mg, p=1.15×10⁻⁶) and C-EDA (123±2 ng/mg, p=4.79×10⁻⁵) segments, respectively (FIG. 21).

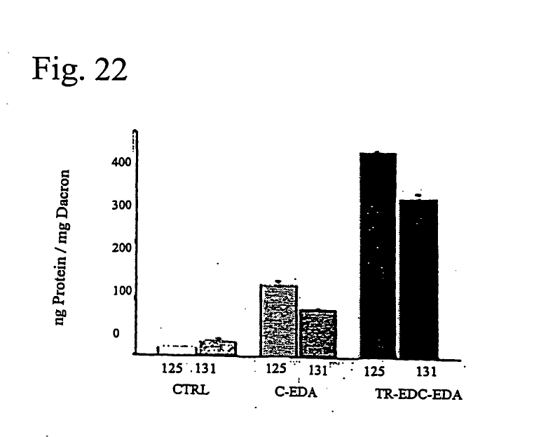

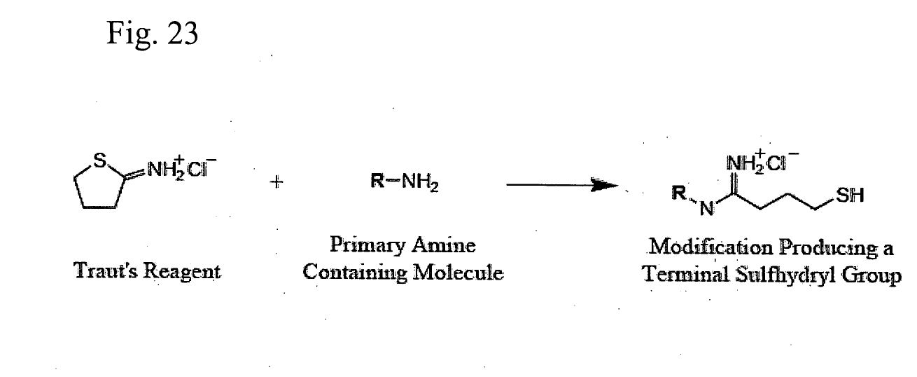

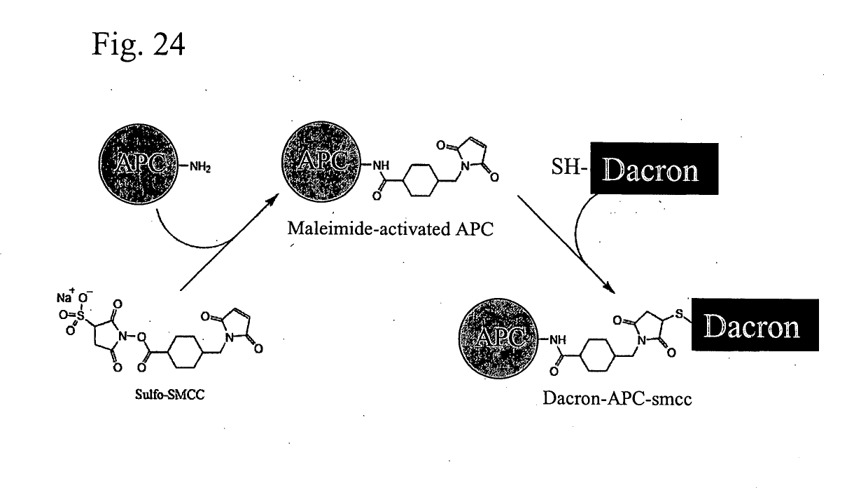

For the double protein binding study, ¹²⁵I-BSA and 131I-BSA binding to the Tr-EDC-EDA segments (367±6 ng/mg and 286±8 ng/mg, respectively) was 26.5 fold and 11.5 fold greater than CTRL segments (14±1 ng/mg, p=1.5×10⁻⁹; 25±5 ng/mg, p=1.37×10⁻⁴, respectively), and 2.9 fold and 3.1 fold greater than the C-EDA segments (127±6 ng/mg, p=1.21×10⁻⁷; 94±5 ng/mg, p=4.5×10⁻⁷, respectively; FIG. 22).

Reaction of EDA with DACRON® provides functional groups within the polymer backbone. These functional groups are accessible for either individual or simultaneous protein binding and can be used for covalent attachment of biologically-active proteins to the DACRON® surface.

### EXAMPLE 4

In Vitro and In Vivo Assessment of Novel Bifunctionalized DACRON® Surfaces

We next sought to evaluate endothelial cell proliferation on DACRON® surfaces modified with the bifunctional amine ethylene diamine (EDA) in vitro and to assess the wound healing response to the modifications in vivo.

In vitro

MYLAR®, a flattened form of DACRON®, was used for in vitro experiments. Discs (1.5 cm diameter) were treated with either 15% NaOH for 30 minutes at 100° C. (HYDRO), EDA for 80 minutes at 25° C. (C-EDA), or a combination of NaOH and EDA (H-EDA) to create functional groups. Human umbilical vein endothelial cells (HUVEC) were then cultured with either complete media or serum-starved media on our modified MYLAR® surfaces. Untreated MYLAR® served as the control (CTRL). HUVECs added to tissue culture wells without MYLAR® were also monitored for cell viability. HUVEC growth was monitored at 1, 2, 3, and 4 days using an Alamar blue assay. Alamar blue interacts with the cell wall of live cells and can be detected at a fluorescence emission spectra of 590 nm.

HUVEC proliferation occurred on all of our modified surfaces throughout the four-day time interval. H-EDA surfaces demonstrated significantly greater HUVEC growth with complete media as compared to the CTRL surfaces (64.05±4.38 vs. 38.25±10.68, p=0.03). C-EDA surfaces demonstrated significantly prolonged HUVEC life with serum-starved media as compared to the CTRL surfaces (3.30±0.51 vs. 1.57±0.28, p=0.005).

In vivo

For the in vivo study, woven DACRON® patches (1 cm²) were treated using the same methods as employed for the MYLAR® discs. The patches were then implanted into a subcutaneous rat model for 14 days, explanted, and wound healing assessed using histological techniques.

Histological evaluation of our explanted patches revealed no impairment or overall difference in wound healing between the modified DACRON® patches and CTRL.

This study demonstrates that covalent attachment of biologically-active agents, such as growth factors, to the accessible functional groups on this DACRON® surface can be used to promote endothelial cell recruitment following, e.g., prosthetic arterial grafting.

### EXAMPLE 5

Immobilization of APC to a biocompatible medical device will render surface antithrombogenecity by renewably inactivating FVa and FVIIIa and by anti-platelet activation and will promote adherence to the device of EPCR positive cells circulating in the blood through a highly specific, high affinity ligand-receptor binding reaction and signal graft-adherent cells or cells from adjacent endothelium to proliferate and migrate on the graft surface.

APC can be immobilized onto the surface of a medical device, such as a Dacron graft surface. Two major properties, the anticoagulant activity and the on surface cell growth, both of which are major factors involved in vascular failure, have been examined and are discussed below.

Creation of Bifunctional (—NH2 and —COOH) Surface

Dacron, the commercial name for polyester, is selected for this study based on its excellent long-term biodurability, handling characteristics, clinical acceptance and potential for chemical manipulation. Dacron materials are not readily reactive with functional molecules like anti-coagulants, growth factors, etc. Thus the first step is to create a surface to which biologically active agents could be covalently attached. Among the multiple choices of surface modification reagent, ethylenediamine (EDA) is selected over alkaline hydrolysis or other active amines for reasons that EDA treatment creates bifunctional groups (—NH2 and —COOH) instead of one and does not significantly alter the physical and chemical properties of the original Dacron material that is important to make it good graft material.

Create APC Anchor Sites (—SH) on ED surface (TED)

Traut's Reagent is used to react with amines on EDA-treated Dacron segments (ED), creating thiol groups (=13 SH) on the Dacron surface. The presence of surface thiol groups on Traut's reagent treated ED (TED) segments is confirmed using Ellman's reagent. The thiol groups are used as anchor sites for APC covalent binding. To trace binding, part of APC is iodinated using carrier free ¹²⁵I and iodobeads. The iodinated APC (¹²⁵I-APC) and free ¹²⁵I are separated by PD-10 desalting column.

APC Activation and Covalent Binding to TED

Sulfo-SMCC is a water-soluble, non-cleavable and membrane impermeable cross-linker. It contains an amine-reactive NHS ester and a thiol-reactive maleimide group. APC is reacted with Sulfo-SMCC, the resulting maleimide-activated APC molecule contains maleimides which are used to react with the thiol-groups on TED surface to achieve covalent binding of APC to TED. The reaction condition is optimized to produce the best covalent linking with minimal loss of function.

Functional Integrity of APC in Solution or Surface Bound

The functional integrity of APC in solution (maleimide-activated APC before covalent linking) and surface bound is evaluated using two methods. The enzymatic activity of APC is examined using a chromogenic assay, which is based on the ability of APC to cleave the specific APC substrate S-2366. The anticoagulant activity of APC is examined using one step clotting assay, which is based on the ability of APC to inhibit blood clot formation. Prolonged clotting time indicates anticoagulant activity.

Biostability of APC in Simulated Flow Systems

The physical and functional biostabilites of surface bound APC are examined using either a static system or a simulated arterial flow system with pulsatile pressure and defined flow rate. A peristaltic pump is used to provide pulsatile flow with flow rate 40 ml/min, pressure 100 mm Hg, temperature at 37° C. The flow environment at the exit site of the graft is monitored by an electromagnetic flowmeter. All experiments are conducted using a 5% BSA/PBS perfusate containing 200 unit/ml penicillin and 0.2 mg/ml streptomycin to prevent bacterial contamination. Priming volume of the system was 400 ml and recirculated. The segments are challenged in the flow system continuously. The physical presence of APC on the surface of the graft is monitored by tracing ¹²⁵I-APC amounts. The functional biostability of surface-bound APC is monitored using a chromogenic assay.

In Vitro Evaluation of Surface Endothelialization

The effect of immobilized APC on surface endothelialization is examined using two in vitro studies. Human coronary artery endothelial cells and human coronary artery smooth muscle cells will be used to examine cellular popularization, proliferation, and migration on Dacron surface in cell culture conditions. The first study involves direct seeding of cells onto Dacron segments with or without immobilized APC and cultured without any disturbance. Low density cell numbers will be seeded, the proliferation of surface grown endothelial cells will be monitored using Alamar blue assay. The second study involves ligand (surface bound APC)-receptor (EPCR on cell surface) binding. Varying numbers of cells will be incubated with segments with or without immobilized APC for a short period of time so that ligand-receptor reaction brings EPCR positive cells onto APC containing surface. Surface bound cells and unbound cells will be separated by removing and washing the segments. The segments will then be placed into a normal culture condition. Alamar blue assay will indicate the initial surface-attached endothelial cell numbers and/or proliferation rate.

Creation of Bifunctional Groups on Dacron Surface

Woven Dacron sheets were washed in scouring solution (1% Na₂CO₃ containing 1% Tween-20), rinsed in dH₂O, air-dried overnight. The bifunctional surface was created by incubation of segments (0.25 cm²) from the washed Dacron with 100% ethylenediamine (EDA) for 20, 40, 80 and 150 min. The segments were removed and placed into dH₂O overnight at room temperature. Methylene blue (MB), was used to determine carboxylic acid groups within the segments. Briefly, the segments were incubated with MB working solution (5 mg/ml MB in 10 mM Tris-HCl, pH 8.0) for 1 hour at room temperature. The segments were removed and washed in 10 mM Tris-HCl buffer for one hour. Segments were then grossly observed for color uptake and shade differences and photographed. Acid red 1 (ARI) was used to assess total (primary and secondary) amine content in the segments. Briefly, segments were incubated with AR-1 working solution (50 mg/ml AR-1 in 10 mM MES, pH 4.5). The segments were removed and washed in MES buffer for one hour. Segments were then grossly observed for color uptake and shade differences, followed by image photography.

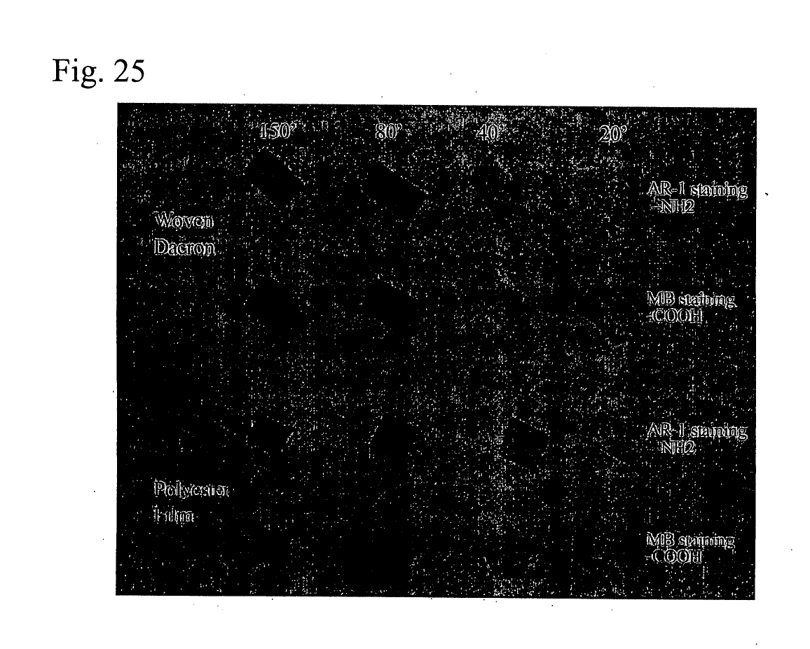

Exposure of the Dacron material to EDA created both carboxylic acid and amine groups within the polymer structure as evidenced by uptake of both methylene blue and acid red. Exposure of segments to EDA for 80 min and 150 min created most and comparable amount of total amine and carboxylic groups (FIG. 25). Thus, all the following Dacron segment materials used were exposed to EDA for 80 min.

Molecular Modification of APC by Sulfo-SMCC and the Functional Evaluation of Modified APC-SMCC

Recombinant human APC was reacted with freshly prepared Sulfo-SMCC at different molar ratio (1:0; 1:2; 1:10 and 1:50) in PBS buffer at 37° C. for 30 min. Purification of the intermediate APC-SMCC complexes form unreacted Sulfo-SMCC was achieved using PD-10 desalting columns equilibrated with PBS. The enzymatic activity of APC-SMCC was evaluated using the specific APC substrate-S2366 (chromogenic assay) and the anticoagulant integrity of APC-SMCC was evaluated using a one step clotting assay. For the chromogenic assay, 50 μl of 0.5 mM S-2366 was incubated with 500 μl of either APC-SMCC or non-modified APC standards at 37° C. for 30 min. The reaction was stopped by adding 2% citric acid. The O.D. 405 nm was read and APC-SMCC activity was read against a standard curve. For the clotting assay, 50 μl of APC-SMCC was added into a pre-warmed test tube, equal volume of pooled normal plasma (either from dog or from human) was added. The clotting was initiated by adding 50 μl of pre-warmed 25 mM CaCl₂. The clotting time (from the moment of adding the CaCl₂ solution to clot formation) was measured.

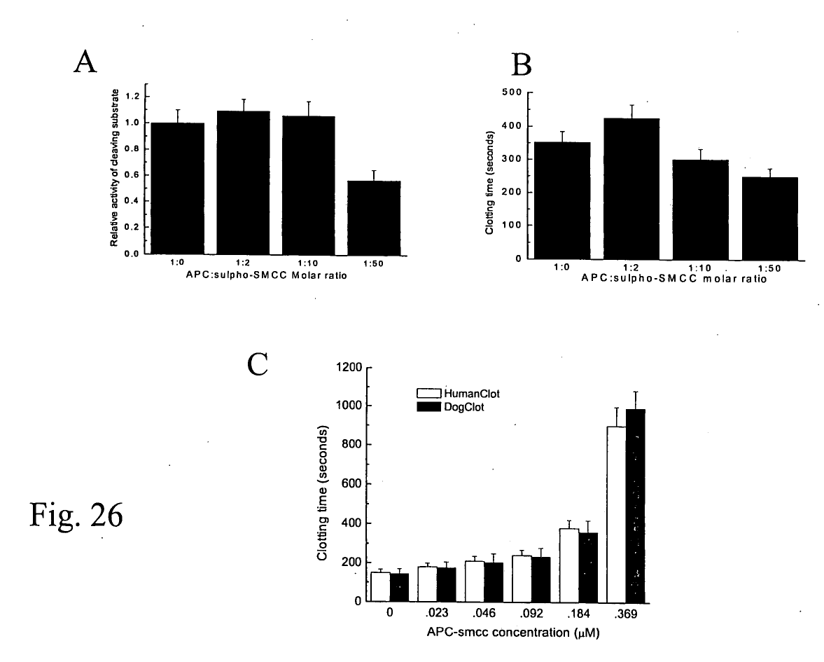

APC:Sulfo-SMCC molar ratio had significant effect on APC-SMCC activity. APC: Sulfo-SMCC molar ratio at 2 had no negative effect on APC activity, while at 50, APC activity was significantly reduced (FIGS. 26A and 26B). Next, we compared the anticoagulant activity of APC-SMCC on dog plasma and human plasma. APC-SMCC appeared to be equally effective in inhibiting clot formation of human plasma and dog plasma (FIG. 26C). This data suggest that a canine vascular bypass is a feasible model for future studies.

Covalent Binding of APC-SMCC to Traut's Reagent Activated EDA-Dacron Surface (TED)

To create thiol groups on material surface, EDA treated Dacron sheet was cut into 0.25 cm² segments and then reacted with 5 mg/ml Traut's reagent in PBS for 1 hour at room temperature. The presence of thiol groups on EDA-Dacron surface was confirmed by reacting with Ellman reagent. TED surface turned yellow, while neither the untreated ED nor the D alone had any color change in Ellman solution. The TED and control segments were then reacted with iodinated APC-SMCC overnight at room temperature on an inversion mixer. The segments were then individually sonicated in 2 ml of PBS with 0.05% Tween-20. This procedure was repeated three times, changing the wash buffer between sonications to remove any weakly adherent APC-SMCC on the surface of each segment. The surface bound radioactivity was then gamma counted and the APC-SMCC bound (ng) per mg Dacron was determined.

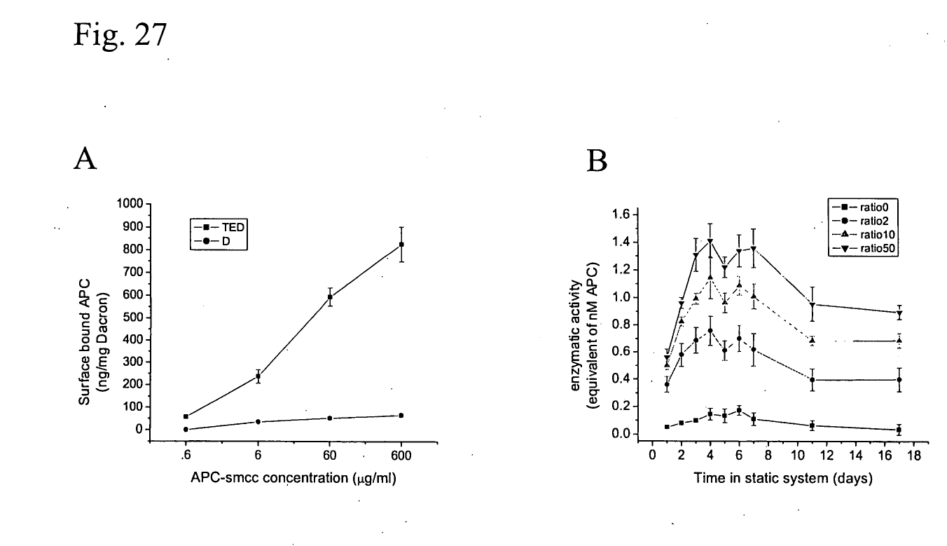

Binding of APC increased with increasing concentration of APC in the reaction solution in both D and TED segments. The TED segments bound at least 10 fold more APC than the D segments at any given APC concentration, suggesting a very high APC binding capacity of TED compared to control Dacron (FIG. 27A). While crosslinker-modified APC activity in solution remained unchanged with SMCC ratio at 1:2 compared to native APC (ratio 1:0), and decreased with ratio greater than 1:10 as indicated in FIGS. 26A and 26B. When surface-bound, APC activity increased with increasing ratio, whether it is due to increased binding is yet to be determined. The substrate cleaving activity continued through the 17 day testing period (FIG. 27B).

Biostability of Surface Bound APC

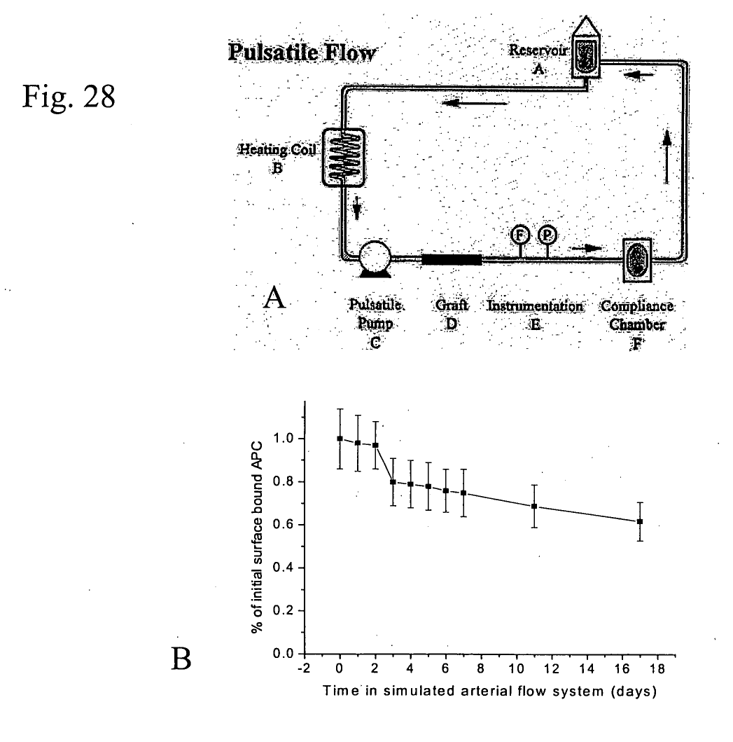

A simulated arterial flow system (FIG. 28A) was used to examine the biostability of surface bound APC. A peristaltic pump was used to provide pulsatile flow with flow rate 40 ml/min, pressure 100 mm Hg, temperature at 37° C. The flow environment at the exit site of the graft was monitored by an electromagnetic flowmeter. All experiments were conducted using a 5% BSA/PBS perfusate containing 200 unit/ml penicillin and 0.2 mg/ml streptomycin to prevent bacterial contamination. Priming volume of the system was 400 ml and recirculated. Under this simulated arterial flow conditions, no major loss of surface-bound APC was seen up to 17 days (FIG. 28B).

Anticoagulant Activity of APC Bonded Surface

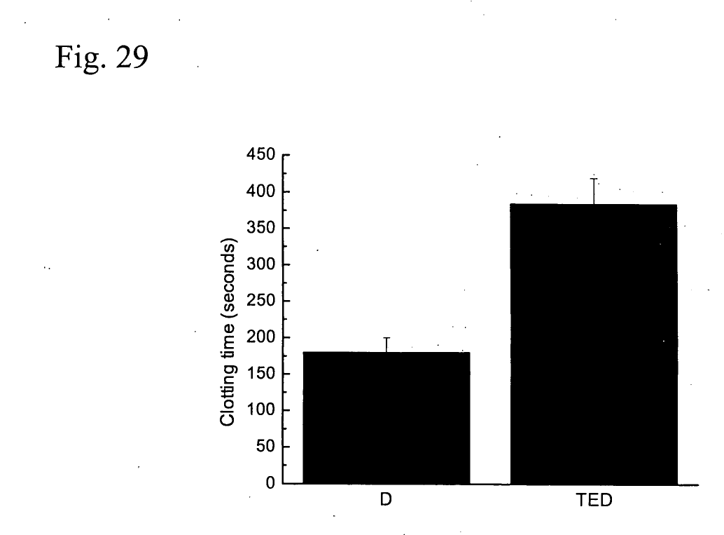

To examine the anticoagulant activity of APC bonded Dacron surface, individual segment (0.5 cm²) was placed into a test tube, and 100 μl of pre-warmed 25 mM CaCl₂ was added into the test tube. To initiate blot clotting, 100 μl of pooled normal human plasma was added and incubated at 37° C. The clotting time (from the moment of adding the plasma to clot formation) was measured. As shown in FIG. 29, covalent linking of APC on TED surface showed significant anticoagulant activity as indicated by the prolonged clotting time compare to control Dacron segments.

Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the appended claims.

Other embodiments are within the claims.

What is claimed is:

1. A biocompatible medical device comprising a coating, wherein said coating comprises activated protein C.

2. The device of claim 1, wherein said device comprises a polyethylene terephthalate polymer comprising a carboxylic acid group derived from a terephthalate moiety of said polymer and an amine group derived from reaction of said polymer with a diamine, wherein said APC is covalently bonded to said polyethylene terephthalate polymer via said carboxylic acid group or said amine group.

3. The device of claim 2, wherein said carboxylic acid group results from hydrolysis of said terephthalate moiety.

4. The device of claim 2, wherein said diamine is selected from ethylene diamine (EDA), 2-methylpentamethylene diamine, 1,2-diaminocyclohexane, and 1,6-hexanediamine.

5. The device of claim 4, wherein said diamine is EDA.

6. The device of claim 5, wherein said polyethylene terephthalate polymer is treated by alkaline hydrolysis prior to reaction with EDA.

7. The device of claim 1, wherein said coating further comprises a chemical compound or a biologically-active agent.

8. The device of claim 7, wherein said chemical compound comprises a commercial finish selected from the group consisting of flame retardants, repellents, antistatic agents, and dyes.

9. The device of claim 7, wherein said biologically-active agent comprises an antimicrobial agent, an antifungal agent, an anti-thrombolytic agent, a thrombolytic agent, an antiviral agent, an antiseptic agent, a growth-promoting agent, or a growth-inhibiting agent.

10. The device of claim 7, wherein said biologically-active agent comprises a peptide, a polypeptide, a nucleic acid molecule, an antibody, or a small molecule.

11. The device of claim 10, wherein said polypeptide comprises anti-thrombin, fibronectin, fibrinogen, vitronectin, collagen, streptokinase, urokinase, tissue plasminogen activator (tPA), vascular endothelial growth factor (VEGF), or gamma-interferon.

12. The device of claim 1, wherein said device is selected from the group consisting of a stent, a catheter, a vascular graft, an artificial heart, a blood filter, a pacemaker lead, a heart valve, and a prosthetic graft.

13. A composition comprising a biocompatible material comprising activated protein C (APC) coated thereto.

14. The composition of claim 13, wherein said APC is coated on an external surface of said composition.

15. The composition of claim 13, wherein said biocompatible material comprises a bifunctionalized polyethylene terephthalate polymer comprising a carboxylic acid group derived from a terephthalate moiety of said polymer and an amine group derived from reaction of said polymer with a diamine, wherein said APC is covalently bonded to said polyethylene terephthalate polymer via said carboxylic acid group or said amine group.

16. The composition of claim 15, wherein said carboxylic acid group results from hydrolysis of said terephthalate moiety.

17. The composition of claim 15, wherein said diamine is selected from ethylene diamine (EDA), 2-methylpentamethylene diamine, 1,2-diaminocyclohexane, and 1,6-hexanediamine.

18. The composition of claim 17, wherein said diamine is EDA.

19. The composition of claim 18, wherein said polyethylene terephthalate polymer is treated by alkaline hydrolysis prior to reaction with EDA.

20. The composition of claim 13, further comprising a chemical compound or a biologically-active agent.

21. The composition of claim 20, wherein said chemical compound comprises a commercial finish selected from the group consisting of flame retardants, repellents, antistatic agents, and dyes.

22. The composition of claim 20, wherein said biologically-active agent comprises an antimicrobial agent, an antifungal agent, an anti-thrombolytic agent, a thrombolytic agent, an antiviral agent, an antiseptic agent, a growth-promoting agent, or a growth-inhibiting agent.

23. The composition of claim 20, wherein said biologically-active agent comprises a peptide, a polypeptide, a nucleic acid molecule, an antibody, or a small molecule.

24. The composition of claim 23, wherein said polypeptide comprises anti-thrombin, fibronectin, fibrinogen, vitronectin, collagen, streptokinase, urokinase, tissue plasminogen activator (tPA), vascular endothelial growth factor (VEGF), or gamma-interferon.

25. The composition of claim 13, wherein said composition is a stent, a catheter, a vascular graft, an artificial heart, a blood filter, a pacemaker lead, a heart valve, a prosthetic graft, or a wound dressing.
